(19) United States
(12) Patent Application Publication
(10) Pub. No.: US 2026/0174963 A1
(43) Pub. Date: Jun. 25, 2026

(54) ASSEMBLY SYSTEM AND RELATED METHODS OF USE

(71) Applicant: Regeneron Pharmaceuticals, Inc., Tarrytown, NY (US)

(72) Inventors: Daniel HALBIG, Ballston Lake, NY (US); Bryan C. GRYGUS, Clifton Park, NY (US); Alexander ROUMANIDAKIS, Schenectady, NY (US); Nicholas VAN PLEW, Selkirk, NY (US)

(73) Assignee: Regeneron Pharmaceuticals, Inc., Tarrytown, NY (US)

(21) Appl. No.: 19/424,381

(22) Filed: Dec. 18, 2025

Related U.S. Application Data
(60) Provisional application No. 63/737,067, filed on Dec. 20, 2024.

(51) Int. Cl. A61M 5/20

(52) U.S. Cl. CPC A61M 5/20; A61M 2207/10

(57) ABSTRACT

In one instance, disclosed herein is a system for assembling an auto-injector including a housing and a shroud, the housing including an opening, and the shroud including a coupling mechanism, the system comprising: a holder configured to control a movement the housing along a first direction; a brace configured to control a movement of the shroud along the first direction; and a pin extending from the brace, wherein in a first configuration, the pin is spaced apart from the housing and the shroud, and in a second configuration, the pin extends through both of the housing and the shroud, the pin configured to maintain the housing and the shroud in contact with one another until the coupling mechanism extends through the opening to couple the housing and the shroud.

### CROSS-REFERENCE TO RELATED APPLICATIONS

This application claims priority to U.S. Provisional Patent Application No. 63/737,067, filed on Dec. 20, 2024, which is hereby incorporated by reference in its entirety.

### FIELD OF DISCLOSURE

Aspects of the present disclosure relate to systems and devices for assembling a device delivering a fluid from a needle into a user using mechanisms, e.g., a pressurized medium, which automatically control injection of the needle and fluid. More specifically, embodiments of the present disclosure relate to systems and methods for assembling an auto-injector.

### INTRODUCTION

In various available systems and method for assembling medical devices, such as auto-injectors, multiple pieces of complex machinery are required. Many methods for assembling auto-injectors require multiple movements by a user assembling the medical device or the system, thus complicating the assembly process.

### BRIEF DESCRIPTION OF THE DRAWINGS

The accompanying drawings, which are incorporated into and constitute a part of this specification, illustrate various exemplary embodiments and, together with the description, serve to explain principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements in various embodiments, other than those specifically shown, are contemplated and are within the scope of the present disclosure.

There are many embodiments described and illustrated herein. The described devices and methods are neither limited to any single aspect nor embodiment thereof, nor to any combinations and/or permutations of such aspects and/or embodiments. Moreover, each of the aspects of the described inventions, and/or embodiments thereof, may be employed alone or in combination with one or more of the other aspects of the described inventions and/or embodiments thereof. For the sake of brevity, certain permutations and combinations are not discussed and/or illustrated separately herein.

FIG. 1 is a perspective view of an auto-injector, according to an example of the disclosure.

FIGS. 2-3 are perspective views of a chassis and a container of the auto-injector of FIG. 1, according to an example of the disclosure.

FIGS. 4A-4D are cross-sectional views of a mandrel assembly, according to an example of the disclosure.

FIGS. 5A and 5B are perspective and cross-sectional views, respectively, of lockout windows, according to an example of the disclosure.

FIGS. 6A-6C are cross-sectional views of the mandrel assembly, according to an example of the disclosure.

FIGS. 7A-7C are schematic views of a drive system, according to an example of the disclosure.

FIG. 8 is a perspective view of a valve assembly, according to an example of the disclosure.

FIGS. 9A and 9B are cross-sectional views of an actuator and an activator, according to an example of the disclosure.

FIG. 10 is a perspective view of an auto-injector, according to an example of the disclosure.

FIGS. 11A and 11B are side views of an actuator and a pivot joint, respectively, according to an example of the disclosure.

FIG. 12 is a perspective view of an activator and a valve assembly, according to an example of the disclosure.

FIG. 13 is a perspective view of a shroud and a chassis, according to an example of the disclosure.

FIGS. 14A and 14B are perspective and cross-sectional views, respectively, of a shroud and a chassis, according to an example of the disclosure.

FIGS. 15A and 15B are perspective and cross-sectional views, respectively, of a shroud and a chassis, according to an example of the disclosure.

FIG. 16 is a cross-sectional view of an auto-injector, according to an example of the disclosure.

FIGS. 17A-17C are side views of an auto-injector, according to an example of the disclosure.

FIGS. 18A and 18B are cross-sectional views of a shroud and a cap, according to an example of the disclosure.

FIGS. 19 and 20 are perspective and side views, respectively, of an assembly system of an auto-injector, according to an example of the disclosure.

FIG. 21 is an enlarged view of cross-sectional view of an assembly system of FIG. 19 of an auto-injector, according to an example of the disclosure.

FIG. 22 is an enlarged view of a section of the assembly system of FIG. 19 before assembly of an auto-injector, according to an example of the disclosure.

FIG. 23 is a partial cross-sectional view of an assembly system before assembly of an auto-injector, according to an example of the disclosure.

FIG. 24A is a partial cross-sectional view of an assembly system before assembly of an auto-injector, according to an example of the disclosure.

FIG. 24B is an enlarged view of a section of the assembly system of FIG. 24A before assembly of an auto-injector, according to an example of the disclosure.

FIG. 25A is a partial cross-sectional view of an assembly system during assembly of an auto-injector, according to an example of the disclosure.

FIG. 25B is an enlarged view of a section of the assembly system of FIG. 25A during assembly of an auto-injector, according to an example of the disclosure.

FIG. 26A is a partial cross-sectional view of an assembly system during assembly of an auto-injector, according to an example of the disclosure.

FIG. 26B is an enlarged view of a section of the assembly system of FIG. 26A during assembly of an auto-injector, according to an example of the disclosure.

FIG. 27A is a partial cross-sectional view of an assembly system during assembly of an auto-injector, according to an example of the disclosure.

FIG. 27B is an enlarged view of a section of the assembly system of FIG. 27A during assembly of an auto-injector, according to an example of the disclosure.

FIG. 28A is a partial cross-sectional view of an assembly system after assembly of an auto-injector, according to an example of the disclosure.

FIG. 28B is an enlarged view of a section of the assembly system of FIG. 28A after assembly of an auto-injector, according to an example of the disclosure.

FIG. 29 is a partial cross-sectional view of an assembly system after assembly of an auto-injector, according to an example of the disclosure.

There are many embodiments described and illustrated herein. The present disclosure is neither limited to any single aspect nor embodiment thereof, nor to any combinations and/or permutations of such aspects and/or embodiments. Each of the aspects of the present disclosure, and/or embodiments thereof, may be employed alone or in combination with one or more of the other aspects of the present disclosure and/or embodiments thereof. For the sake of brevity, many of those combinations and permutations are not discussed separately herein.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the cross-sectional views are not drawn to scale and should not be viewed as representing proportional relationships between different components. The cross-sectional views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. Embodiments of the present disclosure may be used with any type of fluid-containing products, such as liquid drug substances, liquid placebos, or other liquids that may be dispensed in a dose form. In the discussion that follows, terms “about,” “approximately,” “substantially,” and the like, when used in describing a numerical value, denote a variation of +/−10% of that value, unless specified otherwise.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Notably, an embodiment or implementation described herein as an “example” or “exemplary” is not to be construed as preferred or advantageous, for example, over other embodiments or implementations; rather, it is intended reflect or indicate the embodiment(s) is/are one “example,” rather than “ideal.” As used herein, the terms “top,” “bottom,” “upper,” “lower,” “lateral,” and “radial” refer to a location (or portion of a device) relative to an arrangement of the device depicted in the drawings. The terms “vertical,” “vertically,” “horizontal,” “horizontally,” “upwards,” “downwards,” “laterally,” and “radially” refer to a direction or an orientation relative to an arrangement of the device depicted in the drawings. In addition, the terms “first,” “second,” and the like, herein do not denote any order, quantity, or importance, but rather are used to distinguish an element, a structure, a step or a process from another. Moreover, the terms “a” and “an” herein do not denote a limitation of quantity, but rather denote the presence of one or more of the referenced items.

Some conventional auto-injectors may require multiple user interactions to self-administer a drug, including, e.g., separate user interactions for deploying a needle and subsequently retracting the needle after drug delivery. These additional steps can increase complexity of self-administration of drugs, introduce user errors, and cause user discomfort. Accordingly, the present disclosure is directed to various embodiments of an injection device (e.g., auto-injector) that simplifies self-administration of drugs, or other therapeutic agents, by a user. Specifically, according to certain embodiments, the auto-injector may not require any additional user interaction to withdraw a needle once the needle is subcutaneously inserted into the user. Thus, auto-injectors of the present disclosure are simplified to help prevent misuse or user error. Additionally, some conventional auto-injectors require multiple components and user operations to administer a drug, including, various spring or motor mechanisms. These additional components can increase complexity of manufacture and introduce mechanical faults or user error. Accordingly, the present disclosure is directed to various embodiments of an injection device (e.g., auto-injector) that simplifies and refines administration of drugs, or other therapeutic agents. The auto-injectors of the present disclosure may include one or more components that may be substantially similar to those described in International PCT Application No. PCT/US 2020/040729, published as WO 2021/003409, International PCT Application No. PCT/US 2021/065567, published as WO 2022/147166, and International PCT Application No. PCT/US 2024/034746, published as WO 2024/263725, each of which are incorporated by reference.

In at least some embodiments, a handheld auto-injector may require a user to hold the auto-injector against the user's skin for the entirety of an injection procedure. In some embodiments, a handheld auto-injector according to this disclosure may be configured to deliver a medicament volume of less than 3.5 mL (or a medicament volume from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL). The medicament volume may be referred to herein as a “dose.” Furthermore, handheld auto-injectors according to the present disclosure may be configured to complete an injection procedure, as measured from 1) a point at which that the user places the auto-injector onto the skin to 2) a point at which the user removes the auto-injector from the skin after completion of an injection, in less than about 30 seconds, less than about 25 seconds, less than about 20 seconds, less than about 15 seconds, or less than about 10 seconds. As described herein, an early-lift event may occur when the user removes the auto-injector from the skin prior to completion of the injection. The auto-injectors of the present disclosure may be configured and operable to initiate a lockout in response to the occurrence of an early-lift event or a completion of dose delivery, thereby inhibiting further use and/or reactivation of the auto-injector for preserving user safety.

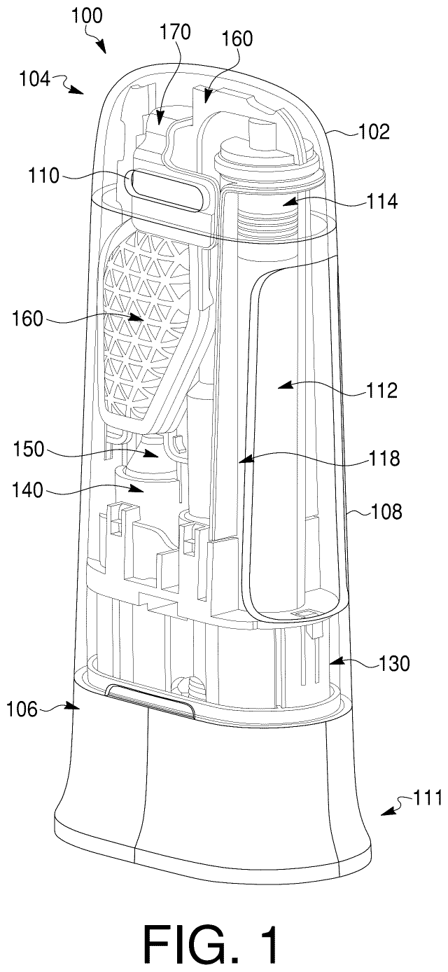

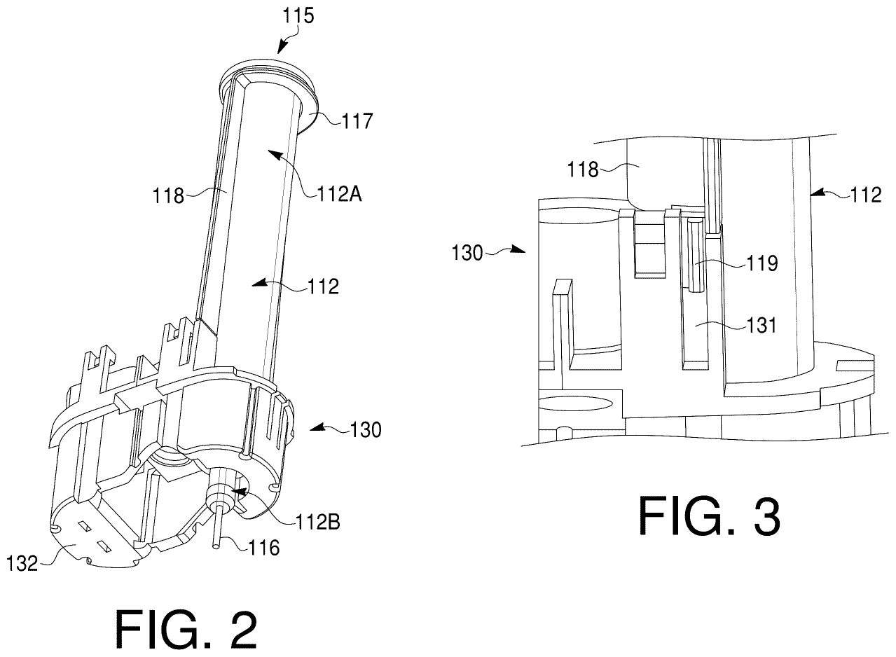

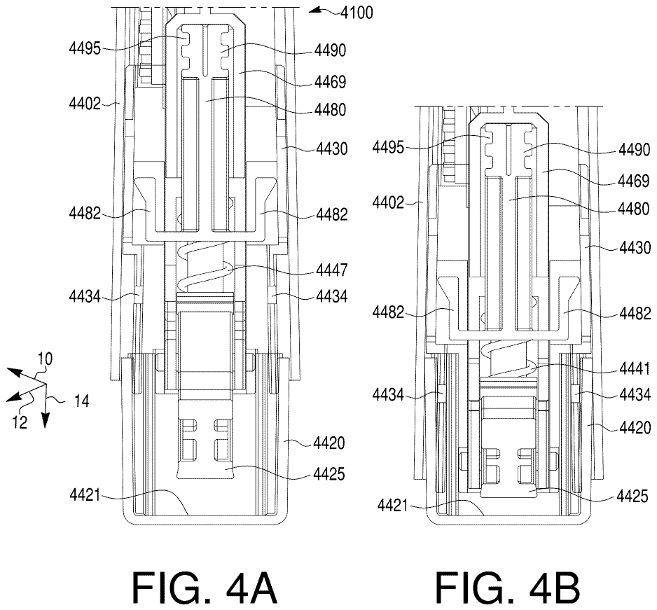

Referring now to FIG. 1, an exemplary auto-injector 100 is depicted in accordance with an example of the present disclosure. Auto-injector 100 may include a housing 102 having a longitudinal length defined between a top end 104 and a bottom end 106, and a cap 111 coupled to bottom end 106. Cap 111 may be removably coupled to bottom end 106, and bottom end 106 may define a user-engaging surface along an exterior interface of bottom end 106 through which a needle (see FIGS. 2-3) may be deployed from and retracted into housing 102. Top end 104 may define a user interface surface from which a user may control auto-injector 100, such as manually grasping housing 102 during use of auto-injector 100. It should be appreciated that housing 102 is depicted in FIG. 1 as partially transparent for illustrative purposes to show the internal components of auto-injector 100.

Auto-injector 100 may include a container 112, a chassis 130, a carrier 140, a canister 150 (e.g., a fluid source), a valve assembly 160, and an indicator assembly 170 housed between top end 104 and bottom end 106 of housing 102. Housing 102 may include one or more windows for facilitating visual inspection of the internal components of auto-injector 100 during use, such as to visually observe a current operating state of auto-injector 100. In the example, housing 102 may include a first window 108 formed along an exterior surface of housing 102 and extending vertically between top end 104 and bottom end 106 along a portion of housing 102 that is aligned with container 112. Accordingly, container 112 and a piston 114 disposed therein may be visually observed from an exterior of housing 102 via first window 108, and specifically the contents of container 112 and a relative position of piston 114 may be visually inspected through first window 108. Auto-injector 100 may include a shield 118 disposed within housing 102 and positioned about container 112 to obstruct visualization of the remaining internal components of auto-injector from first window 108.

Still referring to FIG. 1, container 112 may be sized and shaped to store a nominal value of a medicament. The “nominal volume” (also called the “specified volume,” or “specified capacity”) of a container refers to the container's maximum capacity, as identified by the container's manufacturer or a safety standards organization. A manufacturer or a safety standards organization may specify a container's nominal volume to indicate that the container can be filled with that volume of fluid (either aseptically or not) and be closed, stoppered, sterilized, packaged, transported, and/or used while maintaining container closure integrity, and while maintaining the safety, sterility, and/or aseptic nature of the fluid contained inside. In determining the nominal volume of a container, a manufacturer or a safety standards organization may also take into account variability that occurs during normal filling, closing, stoppering, packaging, transportation, and administration procedures. As an example, a prefillable syringe may be either hand-or machine-filled with up to its nominal volume of fluid, and may then be either vent tube-or vacuum-stoppered, without the filling and stoppering machinery and tools touching and potentially contaminating the contents of the syringe. Alternatively, the stopping machinery and tools may be sterile or aseptic, and are able to contact the contents of the syringe and/or the syringe itself without resulting in any contamination.

Container 112 may have about a 5.0 mL nominal volume in some examples, although any other suitable nominal volume (e.g., from about 0.5 mL to about 50.0 mL, or from about 2.0 mL to about 10.0 mL, or from about 3.0 mL to about 6.0 mL, or from about 1.0 mL to about 3.0 mL, or from about 2.0 mL to about 5.0 mL, or another suitable range) also may be utilized depending on the drug to be delivered. In other examples, container 112 may have a nominal volume greater than or equal to about 0.5mL, or greater than or equal to about 2.0 mL, or greater than or equal to about 3.0 mL, or greater than or equal to about 4.0 mL, or greater than or equal to about 5.0 mL. Container 112 may contain and preserve a drug for injection into a user, and may help maintain sterility of the drug. In one embodiment, container 112 may be configured to deliver a delivered quantity of medicament (e.g., from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL, greater than about 1.0 mL, greater than about 2.0 mL, greater than about 3.0 mL, greater than about 4.0 mL, greater than about 5.0 mL, greater than about 10.0 mL, greater than about 20.0 mL or another delivered quantity).

The delivered quantity may be less than the nominal volume of container 112. Furthermore, in order to deliver the delivered quantity of medicament to a user, container 112 itself may be filled with a different quantity of medicament than the delivered quantity (i.e., a filled quantity). The filled quantity may be an amount of medicament greater than the delivered quantity to account for medicament that cannot be transferred from container 112 to the user due to, e.g., dead space in container 112. Thus, while container 112 may have a nominal volume of 5 mL, the filled quantity and delivered quantity of medicament may be less than 5 mL.

In one embodiment, when container 112 is used in a handheld auto-injector, the delivered quantity of medicament from container 112 may be from about 0.5 mL to about 4.0 mL, about 1.0 mL to about 3.5 mL, about 3.0 mL, about 3.1 mL, about 3.2 mL, about 3.3 mL, about 3.4 mL, about 3.5 mL. The delivered quantity of medicament may be related to the viscosity of the medicament and the hand-held nature of auto-injector 100. That is, in at least some embodiments, at certain viscosities, higher volumes of medicament may prohibit the ability of auto-injector 100 to complete an injection procedure in less than an acceptable amount of time, e.g., less than about 30 seconds. Thus, the delivered quantity of medicament from auto-injector 100 may be set such that an injection procedure, measured from 1) the point in time at which auto-injector 100 is placed onto a user's skin, to 2) the point in time at which auto-injector 100 is removed from the skin, is less than about 30 seconds or less than about another time period (e.g., less than about 25 seconds, less than about 20 seconds, less than about 15 seconds, or less than about 10 seconds).

When the delivered quantity and/or viscosity of the medicament is too high, auto-injector 100 may not be able to function as a handheld auto-injector, since the time required to complete the injection procedure may be higher than commercially or clinically acceptable for handheld devices. Again, as stated above, in embodiments where container 112 is used in a hand-held auto-injector, regardless of the nominal volume of container 112, the delivered quantity of medicament from container 112 may be set such that the injection procedure as defined above is completed in a relatively short period of time (so as to avoid the need for additional features to attach auto-injector 100 to the user so that auto-injector 100 is a wearable auto-injector).

However, it is contemplated that various embodiments of the present disclosure may relate to wearable auto-injectors that deliver relatively large quantities of medicament (e.g., greater than about 3.5 mL) and/or have relatively longer injection procedure times as opposed to handheld auto-injectors (e.g., longer than about 30 seconds, longer than about 1 minute, longer than about 2 minutes, longer than about 5 minutes, or longer than about 1 hour) to complete an injection procedure as measured from 1) the point in time at which the auto-injector is placed onto a user's skin, to 2) the point in time at which the auto-injector is removed from the skin.

Container 112 may have about a 13 mm diameter neck, about a 45 mm length, and an internal diameter of about 19.05 mm. In another embodiment, container 112 may be a standard 3 mL container having an 8 mm crimp top, a 9.7 mm inner diameter, and a 64 mm length. In further embodiments, container 112 may have a length of about 64 mm to 74 mm, such as, for example, about 69.3 mm±0.15 mm (excluding a length of the neck of container 112 at second end 374). In embodiments including the neck, container 112 may have a length ranging from about 65 mm to 75 mm, such as, for example, about 70.8 mm±0.4 mm. These values are merely exemplary, and other suitable dimensions may be utilized as appropriate. In some examples, container 112 may be formed using conventional materials, and may be shorter than existing devices, which can help auto-injector 100 remain cost-effective and small. In some embodiments, container 112 may be a shortened ISO 10 mL cartridge. Auto-injectors of the present disclosure may be configured to deliver highly viscous liquid to a patient. For example, auto-injector 100 of the present disclosure may be configured to deliver liquid having a viscosity from about 0 cP to about 100 cP, from about 5 cP to about 45 cP, from about 10 cP to about 40 cP, from about 15 cP to about 35 cP, from about 20 cP to about 30 cP, or about 25 cP.

Still referring to FIG. 1, container 112 may include a piston 114 movably disposed within a cavity of container 112. Piston 114 may be movable by a pressurized fluid expelled from a fluid source, such as, e.g., canister 150. As described further herein, the pressurized fluid (e.g., gas) expelled from canister 150 may translate piston 114 relative to container 112 vertically along a longitudinal axis of container 112 towards bottom end 112B. The movement of piston 114 towards a bottom end 112B of container 112 may cause the piston to act against the contents within container 112 (e.g., drugs, medications, medicaments, etc.). In some embodiments, auto-injectors of the present disclosure may be oriented such that canister 150 and the piston within container 112 are offset, or are otherwise not longitudinally aligned with one another.

As best seen in FIG. 2, shield 118 may be coaxially coupled onto container 112. Shield 118 may include a top flange 117 that is disposed about and interfaces with a top flange 115 of container 112. Shield 118 may generally have a semi-circular configuration that extends about an exterior surface of container 112 and along a portion of container 112 that is positioned internal to housing 102 and opposite of an opposing portion of container 112 that is positioned adjacent to first window 108 (see FIG. 1). Container 112 may be coupled to chassis 130, and may include a top end 112A adjacent to top flange 115 and a bottom end 112B that is opposite of top end 112A and that extends through a bottom end 132 of chassis 130. Container 112 may include a needle 116 coupled to bottom end 112B, and needle 116 may be in fluid communication with a fluid (e.g., medicament) stored in container 112. As seen in FIG. 3, shield 118 may include a protrusion 119 (e.g. a tab, a finger, etc.) that is received within a slot 131 on chassis 130, thereby coupling shield 118 to chassis 130. In this instance, shield 118 may be fixed to each of container 112 and chassis 130. With protrusion 119 received within slot 131 and top flange 117 coupled to top flange 115, auto-injector 100 may be configured to prevent inadvertent movement (e.g., translation, rotation, etc.) of shield 118 relative to container 112 and chassis 130. Shield 118 may be sized, shaped, and/or otherwise configured to partially cover container 112, thereby inhibiting visual access into an interior of housing 102 via first window 108. In other words, shield 118 may define an opening that is sized, shaped, and/or otherwise configured to reveal container 112 via first window 108 while inhibiting visual exposure of the other components within housing 102. In some embodiments, shield 118 may be formed of an opaque material with a predefined color (e.g., teal) to prevent visual accessibility into housing 102 via first window 108. In other embodiments, shield 118 may be omitted entirely.

Referring back to FIG. 1, housing 102 may further include a second window 110 formed along an exterior surface of housing 102 and extending along a portion of housing 102 that is aligned with a portion of indicator assembly 170. Accordingly, indicator assembly 170 may be visually observed from an exterior of housing 102 via second window 110. As described herein, indicator assembly 170 may be configured to move from a first position towards a second position relative to second window 110 in response to an activation of auto-injector 100, thereby visually indicating a change in operating state of auto-injector 100 through second window 110, and particularly a dose state of auto-injector 100.

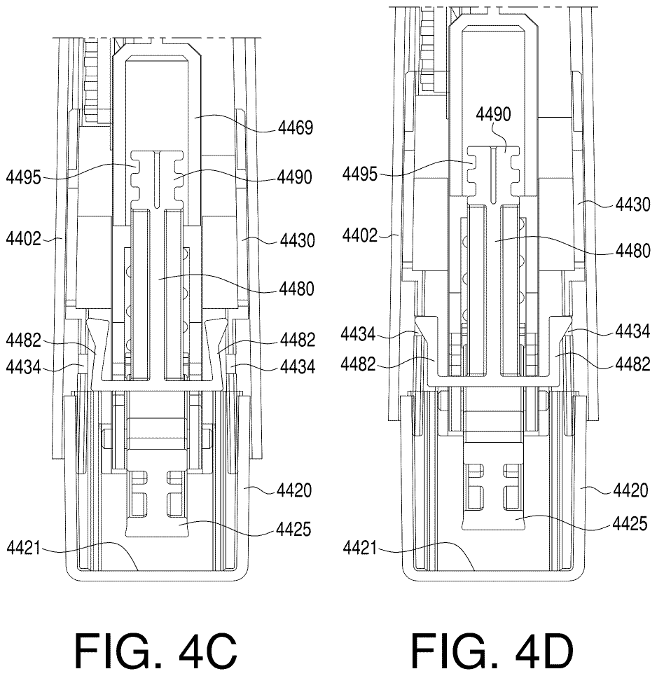

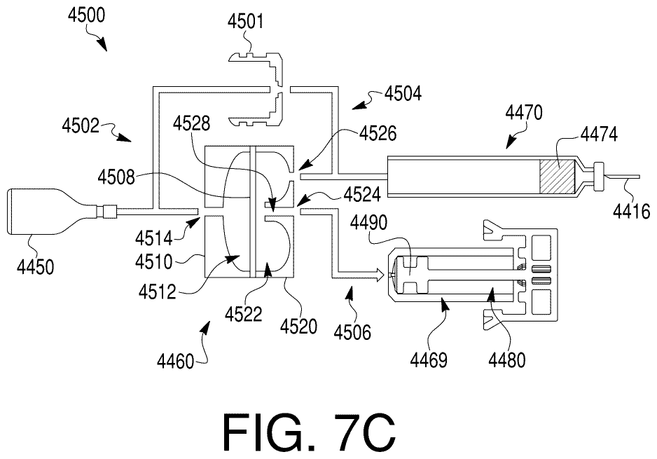

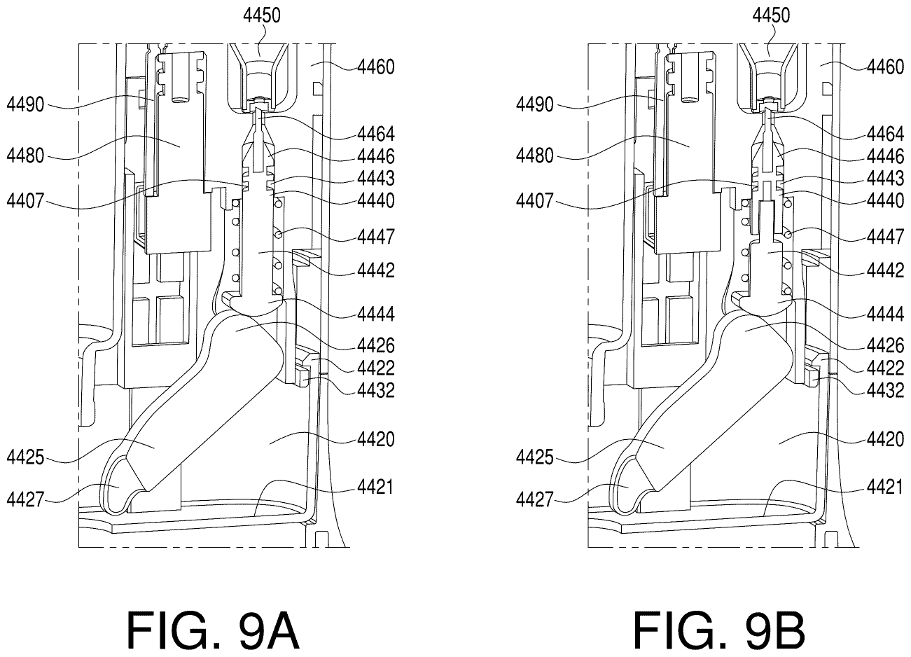

FIGS. 4A-4D depict another exemplary auto-injector 4100, similar to auto-injector 100. Auto-injector 4100 may have any suitable dimensions to enable portability and self-attachment by a user. Another exemplary shroud 4420, chassis 4430, and mandrel assembly 4480 are depicted. It should be appreciated that shroud 4420, chassis 4430, and mandrel assembly 4480 may be incorporated into auto-injector 4100 in a substantially similar manner as shroud 120, chassis 130, and mandrel assembly 180 shown and described above, respectively, except for the differences explicitly noted herein. For example, mandrel assembly 4480 may include a slidable piston 4490. The slidable piston 4490 may be coupled to or integrally formed with mandrel assembly 4480, such that mandrel assembly 4480 and slidable piston 4490 may move together as one unit, e.g., in response to a pressurized medium being released by a canister 4450 (e.g. FIG. 8) through a valve assembly 4460 (e.g. FIG. 8) and into a release outlet 4469 after auto-injector 4100 transitions from an activated state to a post-injection state, as described above and below. After auto-injector 4100 transitions from the activated state to the post-injection state, mandrel assembly 4480 may be configured to engage with shroud 4420, pushing shroud 4420 downward and/or outward relative to the housing 4402 of auto-injector 4100. Mandrel assembly 4480 may further include one or more retention mechanisms 4482. Retention mechanisms 4482 may include, but are not limited to, a clip, a hand, a finger, a tab, a protrusion, a detent, etc. Retention mechanisms 4482 may be configured to engage with one or more lockout windows 4434 included in chassis 4430 as mandrel assembly 4480 pushes downward and/or outward on shroud 4420, allowing mandrel assembly 4480 to lockout shroud 4420 relative to housing 4402, e.g., on or after completing delivery of a dose from auto-injector 4100, to inhibit reactivation or prevent re-exposure of a needle (e.g., needle 4416; FIG. 10).

In the example illustrated in FIGS. 4A-4D, mandrel assembly 4480 may be integrally attached to slidable piston 4490. Mandrel assembly 4480 and slidable piston 4490 may be at least partially disposed within release outlet 4469. Slidable piston 4490 may include one or more recesses 4495 that are sized, shaped, and/or otherwise configured to receive a seal (e.g., an O-ring) therein to inhibit a pressurized medium received by release outlet 4469 from passing along an exterior surface of slidable piston 4490. Accordingly, the pressurized medium received by release outlet 4469 may be operable to exert a downward and/or outward force on slidable piston 4490, thereby causing slidable piston 4490 to translate within release outlet 4469, such as in a downward and/or outward direction, from a first (upper) position, as shown in FIGS. 4A-4B, toward a second (lower) position, as shown in FIG. 4D.

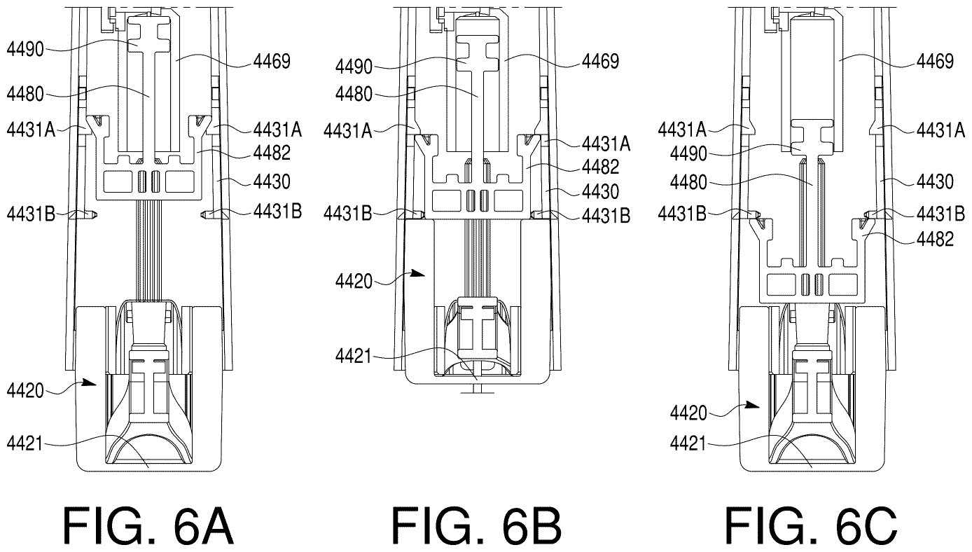

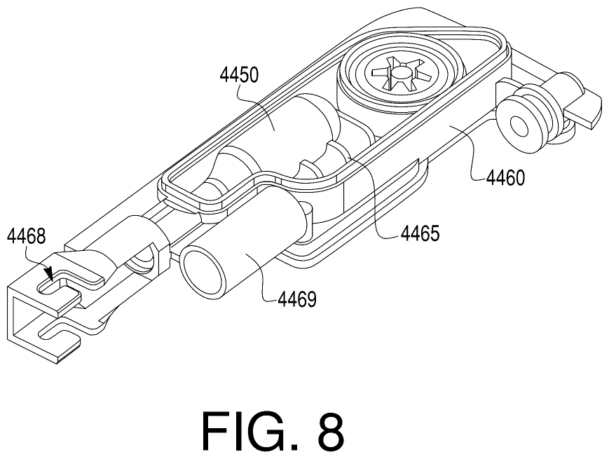

FIG. 4A illustrates auto-injector 4100 in a pre-activation state, e.g., before shroud 4420 has been depressed inward and/or upward relative to chassis 4430 to activate auto-injector 4100. When auto-injector 4100 is in the pre-activation state, shroud 4420 and mandrel assembly 4480 may be in respective first positions. In its respective first position, shroud 4420 may be fully extended downward and/or outward relative to chassis 4430. Referring now to FIGS. 9A and 9B), when auto-injector 4100 is at rest in the pre-activation state, shroud 4420 may be held in its first position by a downward and/or outward force exerted on shroud 4420 by an actuator 4425 engaged with an interior bottom surface 4421 of shroud 4420. For example, the downward and/or outward force exerted on shroud 4420 by actuator 4425 may be a translation of a downward and/or outward force exerted on actuator 4425 by an activator 4440. The downward and/or outward force exerted on actuator 4425 by activator 4440 may be a translation of a downward and/or outward force exerted on activator 4440 by a biasing member 4447 (e.g., a spring) disposed about and configured to engage with activator 4440. In other words, when auto-injector 4100 is in the pre-activation state, biasing member 4447 may be in a compressed configuration, thereby exerting a downward and/or outward force on activator 4440, which may be translated via activator 4440 and/or actuator 4425 onto the interior bottom surface 4421 of shroud 4420, thereby holding shroud 4420 in its respective first position. Additionally or alternatively, when auto-injector 4100 is at rest in the pre-activation state, shroud 4420 may be held in its first position by one or more retaining elements 4422 of shroud 4420 (e.g., one or more lips that protrude from a surface of shroud 4420) configured to engage with one or more corresponding retaining elements 4432 of chassis 4430 (e.g., one or more lips that protrude from a surface of chassis 4430), such that the one or more retaining elements 4422 of shroud 4420 and the one or more corresponding retaining elements 4432 of chassis 4430 prohibit shroud 4420 from being pushed down and/or out of housing 4402. For example, a downward-facing surface of the one or more retaining elements 4422 may be configured to engage an upward-facing surface of the one or more retaining elements 4432. Referring again to FIG. 4A, when mandrel assembly 4480 is in its respective first position, the one or more retention mechanisms 4482 are positioned before the one or more lockout windows 4434 of chassis 4430 along a direction extending from the slidable piston 4490 to the interior bottom surface 4421 of shroud 4420. Mandrel assembly 4480 may be held in its respective first position by friction between one or more seals disposed within the one or more recesses 4495 of slidable piston 4490 and an interior surface of release outlet 4469. Additionally or alternatively, mandrel assembly 4480 and/or slidable piston 4490 may be held in its respective first position at least partially by the engagement between the one or more retention mechanisms 4482 of mandrel assembly 4480 and one or more features of chassis 4430, such as one or more audible feedback elements 4431 (FIG. 6A).

FIG. 4B illustrates auto-injector 4100 in an activated state, e.g., after shroud 4420 has been depressed toward chassis 4430 to activate auto-injector 4100, thereby causing canister 4450 to release a pressurized medium. For example, referring again to FIGS. 9A and 9B, depressing shroud 4420 may cause shroud 4420 to exert an upward and/or inward force on actuator 4425. Actuator 4425 may translate the upward and/or inward force to activator 4440, which may cause a piercing mechanism 4464 (e.g., a needle, an activator pin, etc.) to puncture a seal and/or a valve of canister 4450. When shroud 4420 is depressed, biasing member 4447 may be in a contracted configuration. Referring back to FIG. 4B, shroud 4420 is depicted in a second, fully depressed position. For example, when shroud 4420 is pressed against an exterior surface of a subject's (e.g., a patient) skin, shroud 4420 may be depressed (e.g., translated) upward and/or inward relative to chassis 4430. The upward and/or inward translation of shroud 4420 may be limited by one or more restricting elements (not shown) of housing 4402 or chassis 4430, such that shroud 4420 may be depressed no further than its second, fully depressed position. Shroud 4420 may remain in its second, fully depressed position until shroud 4420 is released, such as by being removed from the exterior surface of the subject's skin. When shroud 4420 is depressed, a needle 4416 (FIG. 10) of auto-injector 4100 may be exposed outside of housing 4402, such as through an opening 4423 (FIG. 10) of shroud 4420, as described above.

FIG. 4C illustrates auto-injector 4100 in a post-activation, pre-lockout state, e.g., after shroud 4420 has been depressed upward and/or inward relative to chassis 4430 to activate auto-injector 4100 and after shroud 4420 has been released, such as by being removed from the exterior surface of a subject's skin. In the post-activation, pre-lockout state, shroud 4420 may be allowed to move from a depressed position (e.g., its second, fully depressed position) back toward to its first, resting position. In the post-activation, pre-lockout state, shroud 4420 may be alternatively or additionally urged back toward its first, resting position by one or more forces generated by one or more elements of auto-injector 4100. For example, upon release of shroud 4420 (e.g., the removal of shroud 4420 from the exterior surface of a subject's skin), biasing member 4447, which may have been put into a further compressed configuration in response to the depression of shroud 4420, may expand back toward its original, less compressed configuration, thereby generating a downward and/or outward force that may be translated and/or applied to shroud 4420 by activator 4440 (FIGS. 9A and 9B) and/or actuator 4425. Or for example, a release of a pressurized medium from canister 4450 (FIG. 8) through valve assembly 4460 (FIG. 8) and into release outlet 4469 may cause the pressurized medium to exert a downward and/or outward force on slidable piston 4490, e.g., in a post-injection state of auto-injector 4100, as described in further detail below. In turn, slidable piston 4490 and mandrel assembly 4480 may translate down and/or out (e.g., within release outlet 4469), eventually engaging with and urging shroud 4420 downward toward its first, resting position.

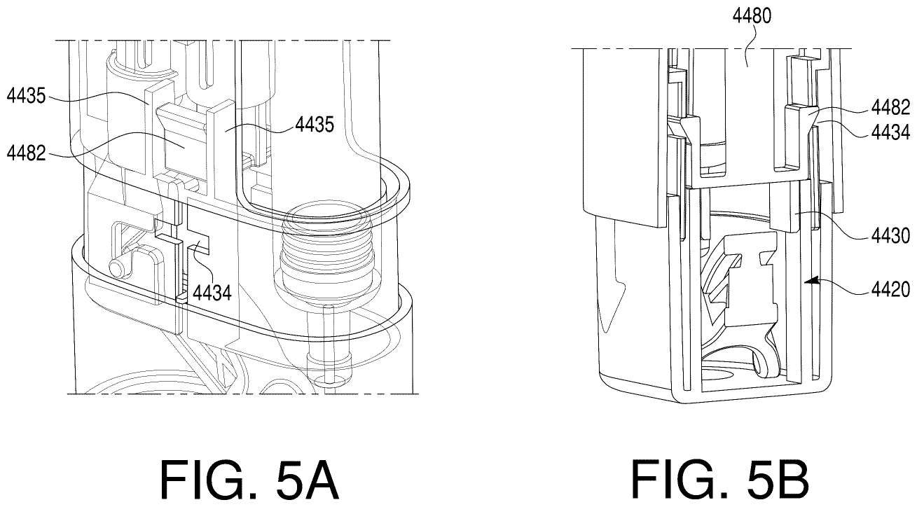

In the example illustrated in FIG. 4A-4D, a width of the interior space of chassis 4430 decreases from an upper portion of chassis 4430 to a lower portion of chassis 4430. Thus, as slidable piston 4490 and mandrel assembly 4480 translate down and/or out in response to the downward and/or outward force exerted by the pressurized medium within release outlet 4469, the one or more retention mechanisms 4482, which are flexible and in contract with the interior wall of chassis 4430, are pressed inward by the decreasing width of the interior space of chassis 4430 (as illustrated in FIG. 4C), until they eventually move into an opening of the one or more lockout windows 4434 of chassis 4430. For example, the one or more lockout windows 4434 may include holes, cutouts, recesses, etc. disposed in the inner and/or outer surface of chassis 4430 and having an opening configured to engage with the one or more retention mechanisms 4482. When the one or more retention mechanisms 4482 move into an opening of the one or more lockout windows 4434, the inwardly pressed retention mechanisms 4482 expand back radially outward and engage with the one or more lockout windows 4434, as illustrated in FIG. 4D. In this example, after the one or more retention mechanisms 4482 engage with the one or more lockout windows 4434, auto-injector 4100 is in a lockout state. As best depicted in FIG. 5B, in the lockout state, the one or more retention mechanisms 4482 and the one or more lockout windows 4434 function cooperatively to prevent shroud 4420 from being depressed, thereby preventing a needle 4416 (FIG. 10) of auto-injector 4100 from being exposed outside of housing 4402 and improving patient safety. For example, the one or more lockout windows 4434 may include a rectangular opening having a flat, downward-facing edge, and the one or more retention mechanisms 4482 may include a flat, upward-facing edge configured to engage with the downward-facing edge of the one or more lockout windows 4434, such that the one or more retention mechanisms 4482, after engaging with the one or more lockout windows 4434, may no longer be translated upward and/or inward.

As depicted in FIG. 5A, chassis 4430 may include one or more guide rails 4435 configured to guide the movement of the one or more retention mechanisms 4482 of mandrel assembly 4480. For example, an interior surface of chassis 4430 may include a pair of guide rails 4435 that are vertical with respect to a longitudinal axis of auto-injector 4100, or otherwise parallel with respect to a longitudinal axis of mandrel assembly 4480, and disposed on either side of a retention mechanism 4482. The pair of guide rails 4435 may be spaced apart at a distance that is slightly greater than a width of the retention mechanism 4482, such that the pair of guide rails 4435 form a slot or track configured to receive and guide the retention mechanism 4482, e.g., as the retention mechanism 4482 moves down and/or out toward the a lockout windows 4434 in a post-activation, pre-lockout state of auto-injector 4100.

As depicted in FIGS. 6A-6C, chassis 4430 may include one or more features or mechanisms configured to generate and provide audible feedback to a user of auto-injector 4100. For example, chassis 4430 may include one or more audible feedback elements 4431 configured to engage with one or more retention mechanisms 4482 of mandrel assembly 4480 to produce an audible sound, e.g., a click. Audible feedback elements 4431 may include bumps, ramps, grooves, divots, holes, or any other element suitable for engaging with retention mechanisms 4482 to produce an audible sound.

In the example depicted in FIGS. 6A-6C, audible feedback elements 4431 include protrusions extending in a radially inward direction away from an interior surface of chassis 4430 and toward mandrel assembly 4480. In this example, mandrel assembly 4480 includes two retention mechanisms 4482, e.g., two arms, in contact with chassis 4430 on opposite sides of mandrel assembly 4480, and chassis 4430 includes two pairs of audible feedback elements 4431, a first pair of audible feedback elements 4431A disposed on opposite interior walls of chassis 4430 along a first horizontal axis 4417 perpendicular to a longitudinal axis 4419 of mandrel assembly 4480, and a second pair of audible feedback elements 4431B disposed on the opposite interior walls of chassis 4430 along a second horizontal axis 4418 perpendicular to the longitudinal axis 4419 of mandrel assembly 4480 and below the first horizontal axis 4417. In a downward direction extending toward the bottom surface 4421 of shroud 4420 along a longitudinal axis of auto-injector 4100, the protrusions of the audible feedback elements 4431 may extend away from the interior surfaces of chassis 4430 to their apexes gradually, such as in the ramped configuration of the first pair of audible feedback elements 4431A, or may extend away from the interior surfaces of chassis 4430 to their apexes virtually instantaneously, such as in the straight configuration of the second pair of audible feedback elements 4431B. In the same direction, beyond their apexes, the protrusions of the audible feedback elements 4431 may retract to the interior surfaces of chassis 4430 instantaneously.

FIG. 6A depicts shroud 4420, chassis 4430, and mandrel assembly 4480 when auto-injector 4100 is in a pre-activation state, e.g., before shroud 4420 has been depressed upward and/or inward relative to chassis 4430 to activate auto-injector 4100. In this example, when auto-injector 4100 is in the pre-activation state, the two retention mechanisms 4482 of mandrel assembly 4480 are engaged with the first pair of audible feedback elements 4431A, which may at least partially hold mandrel assembly 4480 and/or slidable piston 4490 in its respective first position. Thus, when a pressurized medium received by release outlet 4469 exerts a downward and/or outward force on slidable piston 4490, e.g., in a post injection state of auto-injector 4100, as described above, the retention mechanisms 4482 of mandrel assembly 4480 immediately begin moving against the first pair of audible feedback elements 4431A. Because of the ramped configuration of retention mechanisms 4482 and/or the ramped configuration of audible feedback elements 4431, as well as the flexibility of retention mechanisms 4482, as slidable piston 4490 and mandrel assembly 4480 move downward and/or outward, the retention mechanisms 4482 are pressed inward by the first pair of audible feedback elements 4431, until they eventually move beyond the apexes of the first pair of audible feedback elements 4431A. Because the audible feedback elements 4431 retract to the interior surfaces of chassis 4430 instantaneously, when the retention mechanisms 4482 move beyond the apexes of the first pair of audible feedback elements 4431A, the retention mechanisms 4482 immediately expand back radially outward to contact the interior surfaces of chassis 4430, as depicted in FIG. 6B, thereby producing a first audible click. As this first audible click may be produced immediately after auto-injector 4100 transitions from the activated state to the post-injection state, as described in further detail above and below, the first audible click may serve as an audible indication to a user of auto-injector 4100 that an injection from auto-injector 4100 has finished.

FIG. 6B depicts shroud 4420, chassis 4430, and mandrel assembly 4480 when auto-injector 4100 is in a post-activation, pre-lockout state, e.g., after shroud 4420 has been depressed upward and/or inward relative to chassis 4430. In the example, after the retention mechanisms 4482 move beyond the first pair of audible feedback elements 4431A, through further downward and/or outward translation of slidable piston 4490 and mandrel assembly 4480 in response to a release of a pressurized medium into release outlet 4469, mandrel assembly 4480 may be allowed to engage with shroud 4420 and urge shroud 4420 downward and/or outward. As mandrel assembly 4480 and shroud 4420 move downward and/or outward, the retention mechanisms 4482 of mandrel assembly 4480 eventually engage with the second pair of audible feedback elements 4431B, and the retention mechanisms 4482 are pressed inward by the second pair of audible feedback elements 4431B, until they eventually move beyond the apexes of the second pair of audible feedback elements 4431B. Because the audible feedback elements 4431 retract to the interior surfaces of chassis 4430 instantaneously, when the retention mechanisms 4482 move beyond the apexes of the second pair of audible feedback elements 4431B, the retention mechanisms immediately expand back radially outward to contact the interior surfaces of chassis 4430, as depicted in FIG. 6C, thereby producing a second audible click. As this second audible click may be produced at the same time that shroud 4420 returns to its first, resting position, the second audible click may serve as an audible indication that shroud 4420 has been locked out.

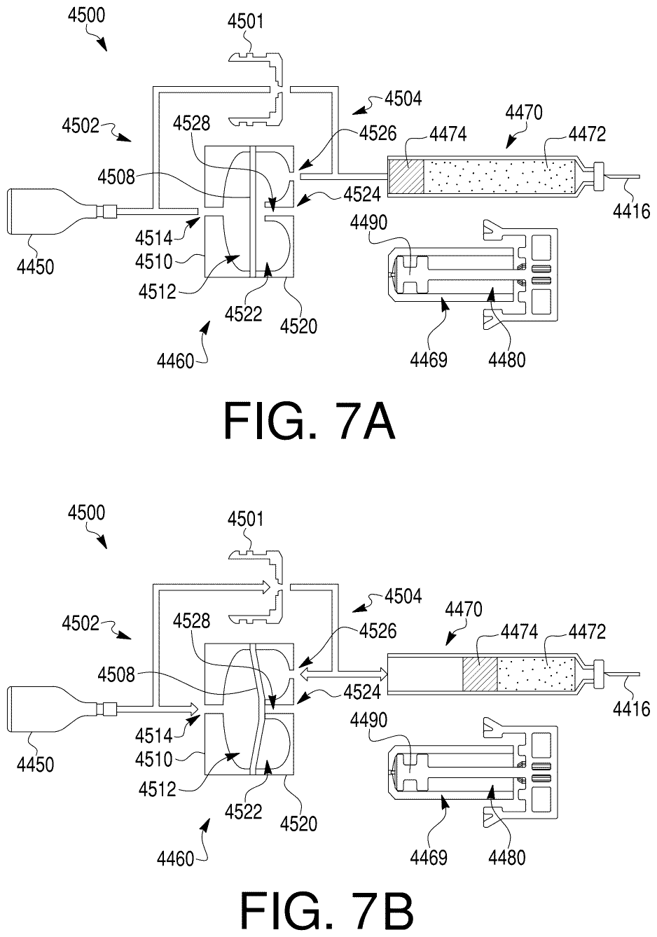

Referring now to FIGS. 7A-7C, an exemplary drive system 4500 of auto-injector 4100 is schematically depicted. Drive system 4500 may be configured to provide a driving force to deliver a medicament 4472 from container 4470 to a patient. As depicted in FIGS. 7A-7C, drive system 4500 may include a canister 4450 containing a pressurized fluid and one or more components of auto-injector 4100 operatively coupled to canister 4450, e.g., valve assembly 4460, container 4470, and release outlet 4469. Drive system 4500 may further include a first flow path 4502, a second flow path 4504, and a third flow path 4506. Each of flow paths 4502, 4504, and 4506 may include one or more lines through which a pressurized fluid may be directed. Canister 4450 may be operatively coupled to, e.g., in fluid communication with, one or more other components of auto-injector 4100, e.g., valve assembly 4460, container 4470, and release outlet 4469, via first flow path 4502, second flow path 4504, and third flow path 4506. For example, canister 4450 may be in fluid communication with valve assembly 4460 via the first flow path 4502; canister 4450 may be in fluid communication with container 4470 via the first flow path 4502 and the second flow path 4504; and canister 4750 may be in fluid communication with release outlet 4469 via the first flow path 4502, the second flow path 4504, and the third flow path 4506. As depicted in FIGS. 7A-7C, drive system 4500 may further include one or more flow restrictors or orifices 4501 disposed about, within, or between first flow path 4502, second flow path 4504, and third flow path 4506.

As depicted in FIGS. 7A-7C, valve assembly 4460 may include a first or high pressure body portion 4510 including a first or high pressure cavity 4512 and a first or high pressure inlet 4514. High pressure cavity 4512 may be in fluid communication with canister 4450 via the first flow path 4502 and high pressure inlet 4514. Valve assembly 4460 may further include a second or low pressure body portion 4520 including a second or low pressure cavity 4522, a second or low pressure inlet 4524, a conduit 4526, and a valve seat 4528. Low pressure cavity 4522 may be in fluid communication with canister 4450 via the first flow path 4502, the second flow path 4504, and the low pressure inlet 4524. Conduit 4526 may be formed within the valve seat 4528, which may extend into the interior of valve assembly 4460, and in particular into low pressure cavity 4522. High pressure cavity 4512 and low pressure cavity 4522 may be defined and fluidly separated by a diaphragm 4508.

FIG. 7A depicts components of drive system 4500 when auto-injector 4100 is in a pre-activated state. As described above and below, in the pre-activated state, shroud 4420 has not yet been depressed to activate auto-injector 4100. Thus, in the pre-activated state, pressurized fluid is not yet released from canister 4450 and the pressure within high pressure cavity 4512 is substantially equal to the pressure within low pressure cavity 4522; piston 4474 is at rest and medicament 4472 is not yet expelled from container 4470; and mandrel assembly 4480 is at rest.

FIG. 7B depicts components of drive system 4500 when auto-injector 4100 is in a post-activation, pre-lockout state. As described above and below, in the post-activation, pre-lockout state, shroud 4420 has been depressed to cause a pressurized fluid to be released by canister 4450, thereby activating auto-injector 4100. As depicted in FIG. 7B, when the pressurized fluid is released by canister 4450, the pressurized fluid begins flowing through the drive system 4500 via the first flow path 4502 and the second flow path 4504. In this example, an orifice 4501 disposed between the first flow path 4502 and the second flow path 4504 restricts the flow of the pressurized fluid between the first flow path 4502 and the second flow path 4504, causing the pressure within the first flow path 4502 to increase more quickly than the pressure within the second flow path 4504, thereby generating a pressure differential between the first flow path 4502 and the second flow path 4504, and, by extension, between the high pressure cavity 4512 and the low pressure cavity 4522. The pressure differential between the high pressure cavity 4512 and the low pressure cavity 4522 in turn causes the diaphragm to move in a first direction toward conduit 4526, thereby sealing valve seat 4528. With the valve seat 4528 sealed, the pressurized fluid cannot flow into the third flow path 4504, and thus the pressure within the second flow path 4502 continues to increase until the pressurized fluid applies enough force to piston 4474 to translate piston 4474 toward needle 4416, thereby expelling medicament 4472 from container 4470. While piston 4474 is translated toward needle 4416, the pressure within the second flow path 4504 may remain substantially constant and/or less than the pressure within the first flow path 4502.

FIG. 7C depicts components of drive system 4500 when auto-injector 4100 is in a post-injection, pre-lockout state. Auto-injector 4100 may be in a post-injection, pre-lockout state when piston 4474 may be translated no further toward needle 4416 by the force exerted on piston 4474 by the pressurized fluid, e.g., when piston 4474 bottoms out within container 4470. As depicted in FIG. 7C, when piston 4474 may be translated no further toward needle 4416 by the force exerted on piston 4474 by the pressurized fluid, the pressure within the second flow path 4504 increases, thereby decreasing the pressure differential between first flow path 4502 and the second flow path 4504, and, by extension, the pressure differential between the high pressure cavity 4512 and the low pressure cavity 4522, until the pressure differential between the high pressure cavity 4512 and the low pressure cavity 4522 reaches a threshold pressure differential low enough to cause diaphragm 4508 to move in a second direction opposite the first direction, thereby unsealing the valve seat 4528. With the valve seat 4528 unsealed, the pressurized fluid may flow through the third flow path 4506 and into release outlet 4469. With pressurized fluid flowing through the third path 4506, the pressure within the third flow path 4506 increases until the pressurized fluid exerts enough force on slidable piston 4490 to translate slidable piston 4490 and mandrel assembly 4480 toward shroud 4420, such that mandrel assembly 4480 may engage with and urge shroud 4420 back toward a third and final lockout position (which may be similar its first, resting position, as described above). While slidable piston 4490 is translated toward shroud 4420 and/or while mandrel assembly 4480 is urging shroud 4420 toward its first, resting position, the pressure within the third flow path 4506 may remain substantially constant. When slidable piston 4490 may be translated no further, e.g., when shroud 4420 has been returned to its first, resting position by mandrel assembly 4480, pressurized fluid within the drive system 4500 may be redirected toward a venting system configured to vent drive system 4500 by releasing pressurized fluid into an interior cavity of auto-injector 4100 and/or into the atmosphere outside of auto-injector 4100, as described above.

As illustrated in FIG. 8, valve assembly 4460 may be configured such that canister 4450 is disposed within valve assembly 4460. Disposing canister 4450 within valve assembly 4460 may allow valve assembly 4460 to occupy a relatively larger space within housing 4402 of auto-injector 4100 than when canister 4450 is disposed outside of valve assembly 4460. Occupying a relatively larger space within housing 4402 of auto-injector 4100 may in turn allow valve assembly 4460 to have a relatively larger volume, which may reduce stress on the valve assembly 4460 and/or increase a duration of an injection provided by auto-injector 4100. Valve assembly 4460 may include one or more retention elements 4465 configured to retain canister 4450 at a particular position within valve assembly 4460. When compare to disposing canister 4450 outside of valve assembly 4460, disposing canister 4450 within valve assembly 4460 may reduce a force (e.g., a blowback force) applied to activator 4440, actuator 4425, and/or shroud 4420 by a pressurized medium released from canister 4450. Reducing the blowback force may in turn reduce a force (e.g., a hold force) required for a user to keep shroud 4420 in its second position while medicament 4472 is released from container 4470 via needle 4416.

FIGS. 9A and 9B illustrate cross-sectional views of auto-injector 4100. As mentioned above, auto-injector 4100 may include a canister 4450 containing a pressurized medium and a valve assembly 4460 configured to receive the pressurized medium when the pressurized medium is released from canister 4450. As mentioned above, canister 4450 may be configured to be punctured by a piercing mechanism 4464 (e.g., a needle, an activator pin, etc.). Piercing mechanism 4464 may include or be included in an activator 4440. Activator 4440 may include a body 4442 and a head 4444. Body 4442 may include a slidable piston 4446 at least partially disposed within activator chamber 4443. Slidable piston 4446 may include one or more recesses 4407 that are sized, shaped, and/or otherwise configured to receive a seal (e.g., an O-ring) therein to inhibit a pressurized medium released by canister 4450 and received by activator chamber 4443 from passing along an exterior surface of slidable piston 4446. A biasing member 4447 (e.g., a spring) may be disposed about body 4442 and configured to engage with activator 4440, such as by exerting a downward and/or outward force on head 4444. Head 4444 may be disposed at an end of activator 4440 opposite piercing mechanism 4464 and may be configured to engage with actuator 4425 (e.g., a movable lever), such that a force exerted on activator 4440 may be translated to actuator 4425 and vice versa. Actuator 4425 may include a first end 4426 configured to engage with head 4444 of activator 4440 and a second end 4427 configured to engage with shroud 4420 (e.g., by abutting against an interior bottom surface 4421 of shroud 4420). Actuator 4425 may be configured to engage with shroud 4420 and activator 4440 simultaneously, such that actuator 4425 may be in operative contact with both shroud 4420 and activator 4440 in any state of auto-injector 4100 (e.g., a pre-activation state, an activated state, a post-activation state, a lockout state, or a completion state).

When auto-injector 4100 is in a pre-activation state, biasing member 4447 may be in a first compressed configuration, thereby generating and applying a downward and/or outward force on head 4444 of activator 4440. In turn, head 4444 of activator 4440 may translate the downward and/or outward force onto first end 4426 of actuator 4425, which may further translate the downward and/or outward force onto shroud 4420, thereby providing resistance to shroud 4420 being depressed upward and/or inward relative to housing 4402. Providing resistance to shroud 4420 being depressed upward and/or inward when auto-injector 4100 is in the pre-activation state may help prevent premature activation of auto-injector 4100 and/or may help prevent a needle 4416 of auto-injector 4100 from being exposed outside of auto-injector 4100 through an opening 4423 of shroud 4420, thereby improving operation of auto-injector 4100 and/or patient safety. Providing resistance to shroud 4420 being depressed upward and/or inward when auto-injector 4100 is in the pre-activation state may be referred to as providing passive needle coverage.

Thus, auto-injector 4100 may include a first mechanism (e.g., biasing member 4447 and/or actuator 4425) configured to urge shroud 4420 toward its first position before auto-injector 4100 is activated and a second mechanism (e.g., mandrel assembly 4480, slidable piston 4490, valve assembly 4460, canister 4450, retention mechanisms 4482, and/or lockout windows 4434) configured to urge shroud 4420 toward its first position and/or inhibit shroud 4420 from moving toward its second position after auto-injector 4100 has been activated (e.g., after a pressurized medium has been released by canister 4450 and/or after medicament 4472 has been released outside of housing 4402). It will be understood that by exerting a downward and/or outward force on shroud 4420, biasing member 4447 may provide resistance to shroud 4420 being depressed upward and/or inward when auto-injector 4100 is any state, not only when auto-injector 4100 is in the pre-activation state. It will be further understood that although auto-injector 4100 is often described herein as employing a pressurized medium to expel medicament 4472 from container 4470 and/or actuate mandrel assembly 4480, auto-injector 4100 may include a first mechanism configured to urge shroud 4420 toward its first position before auto-injector 4100 is activated and a second mechanism configured to urge shroud 4420 toward its first position and/or inhibit shroud 4420 from moving toward its second position after auto-injector 4100 has been activated (e.g., after medicament 4472 has been released outside of housing 4402), while employing any other appropriate driving force for expelling medicament 4472 from container 4470, such as a spring or a motor.

When shroud 4420 is intentionally depressed to activate auto-injector 4100, such as when shroud 4420 is pressed against the exterior surface of a subject's skin, shroud 4420 may exert an upward and/or inward force on second end 4427 of actuator 4425, which may translate the upward and/or inward force onto activator 4440 (e.g., through first end 4426), thereby translating slidable piston 4446 upward and/or inward toward canister 4450, such that piercing mechanism 4464 may puncture a seal or valve of canister 4450 and activate auto-injector 4100, as described above. For example, actuator 4425 may be rotatable about a pivot joint 4468 (FIGS. 8 and 10), such that an upward and/or inward force exerted on second end 4427 causes actuator 4425 to rotate about pivot joint 4468 in a clockwise direction (relative to the perspective of FIGS. 9A and 9B). In this example, as actuator 4425 rotates in the clockwise direction, the point of first end 4426 at which actuator 4425 contacts head 4444 of activator 4440 moves upward and/or inward relative to housing 4402, thereby pushing activator 4440 upward and/or inward, until piercing mechanism 4464 punctures a seal or valve of canister 4450.

As illustrated in FIG. 9B, activator 4440 may be a two-part activator, including a first part including a slidable piston 4446 and a piercing mechanism 4464 and a second part including head 4444. The first part may be configured to receive the second part, but may not be integrally formed with the second part. In such an embodiment, biasing member 4447 may be configured to exert a downward and/or outward force on head 4444 independent of slidable piston 4446, which may aid slidable piston 4446 in preventing a pressurized medium released by canister 4450 and received by activator chamber 4443 from passing along an exterior surface of slidable piston 4446 and/or aid biasing member 4447 in providing passive needle coverage by preventing friction generated between one or more seals (e.g., O-rings) disposed within one or more recesses 4407 and an interior surface of activator chamber 4443 from against biasing member 4447.

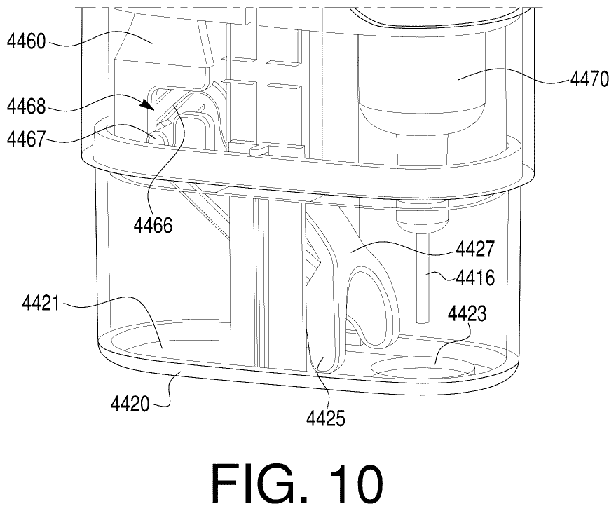

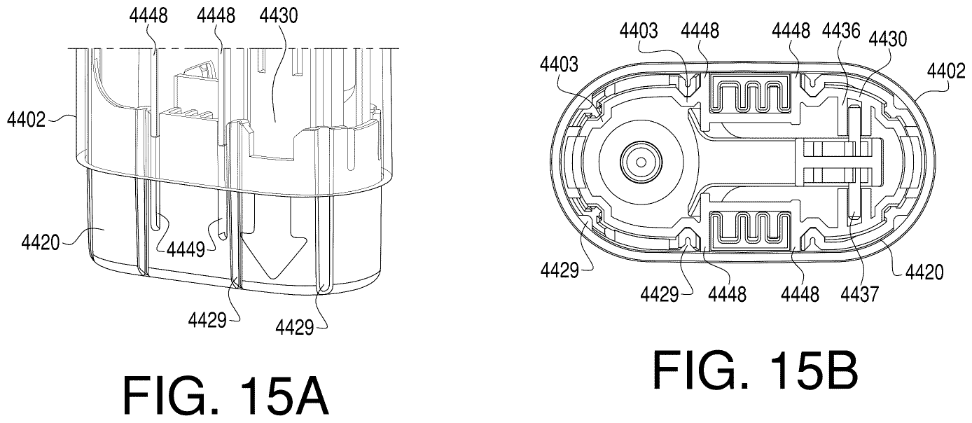

As illustrated in FIG. 10, second end 4427 of actuator 4425 may have a two-pronged form configured to simultaneously contact shroud 4420 (e.g., an interior bottom surface 4421 of shroud 4420) at two points. The two-pronged form of second end 4427 may be configured to straddle an opening 4423 of shroud 4420 as shroud 4420 is depressed toward its second position or as shroud 4420 is urged back toward its first position. Actuator 4425 may be configured to rotate within a pivot joint 4468, e.g., in response to an upward and/or inward force exerted on actuator 4425 by shroud 4420, or in response to a downward and/or outward force exerted on actuator 4425 by activator 4440, as described above. In some instances, as illustrated in FIG. 10, pivot joint 4468 may include one or more pins 4467 projecting outwardly from actuator 4425 and one or more slots or sockets 4466 configured to receive the one or more pins 4467. The one or more pins 4467 projecting outwardly from actuator 4425 may function as an axle about which actuator 4425 may rotate within the one or more slots or sockets 4466. As depicted in FIG. 10, the one or more slots or sockets 4466 may be included in valve assembly 4460. Alternatively, some instances, as depicted in FIGS. 11A and 11B, pivot joint 4438 may include an axle 4437 that actuator 4425 can be affixed to, such as by being snapped onto, and one or more slots or sockets 4436. The axle 4437 may be affixed to pivot joint 4438 via one or more slots or sockets 4436, such as by being snapped into the one or more slots or sockets 4436. The one or more slots or sockets 4436 may include one or more stubs 4439 configured to secure axle 4437 in place after axle 4437 is affixed to the one or more slots or sockets 4436. Once actuator 4425 is affixed to axle 4437 and axle 4437 is affixed to pivot joint 4438, axle 4437 and actuator 4425 can rotate together within the one or more slots or sockets 4436, e.g., in response to an upward and/or inward force exerted on actuator 4425 by shroud 4420, or in response to a downward and/or outward force exerted on actuator 4425 by activator 4440, as described above. As depicted in FIGS. 11B and 15B, the one or more slots or sockets 4436 may be included in chassis 4430.

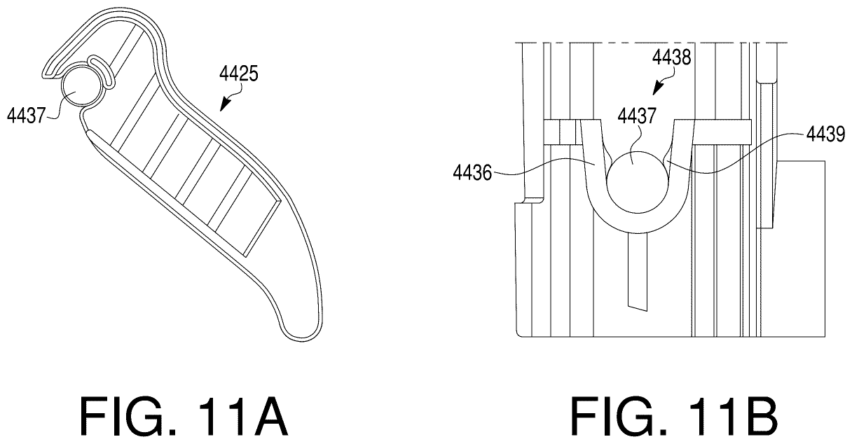

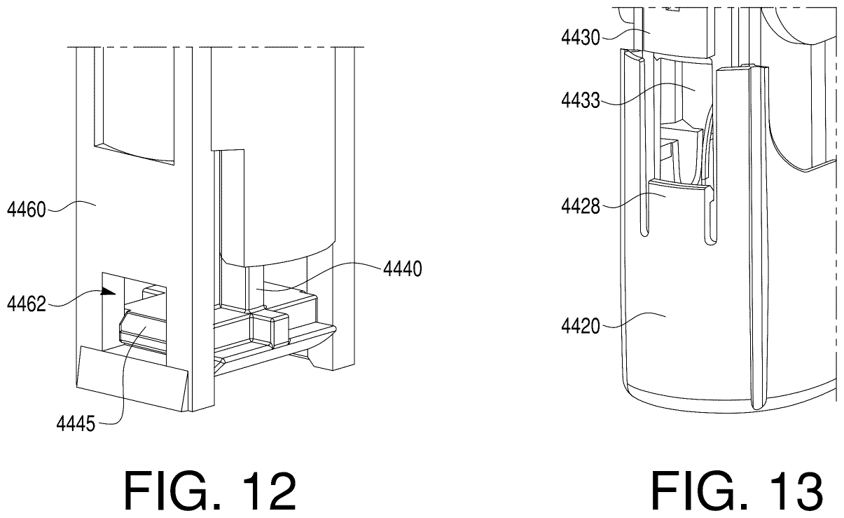

As depicted in FIG. 12, valve assembly 4460 may include one or more retention windows 4462 configured to limit the downward and/or outward translation of activator 4440, e.g., in response to a downward and/or outward force exerted on activator 4440 by biasing member 4447 (FIG. 9B), as described above. For example, activator 4440 may include one or more tabs 4445, and the one or more retention windows 4462 may be openings formed within walls of valve assembly 4460 that are configured to receive the one or more tabs 4445, such that a tab 4445 may be allowed to move up and/or down (e.g., in and/or out) within a retention window 4462, but may be prevented from moving up and/or inward beyond a top border of the retention window 4462 or from moving down and/or outward beyond a bottom border of the retention window 4462. Similarly, as depicted in FIG. 13, chassis 4430 may include one or more retention windows 4433 configured to limit the downward and/or outward translation of shroud 4420, e.g., in response to a downward and/or outward force exerted on shroud 4420 by mandrel assembly 4480 or by actuator 4425, as described above. For example, shroud 4420 may include one or more tabs 4428, and the one or more retention windows 4433 may be openings formed within walls of chassis 4430 that are configured to receive the one or more tabs 4428, such that a tab 4428 (and, by extension, shroud 4420) may be allowed to move up and/or down (e.g., in and/or out) within a retention window 4433, but may be prevented from moving up and/or inward beyond a top border of the retention window 4433 or from moving down and/or outward beyond a bottom border of the retention window 4433.

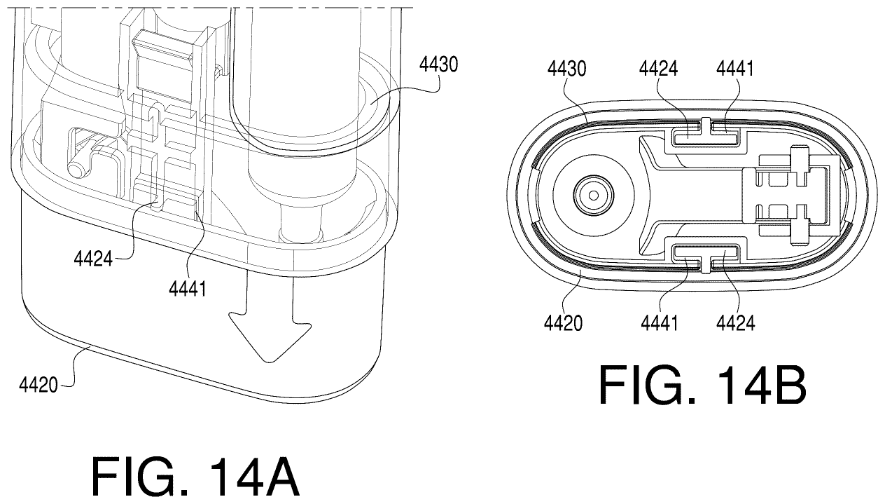

As depicted in FIGS. 14A and 14B, chassis 4430 may include one or more guide rails 4441 configured to guide the movement of shroud 4420. For example, shroud 4420 may include one or more projections 4424, and chassis 4430 may include one or more guide rails 4441 configured to receive the one or more projections 4424 and guide the one or more projections 4424 as shroud 4420 moves up or down (e.g., in or out) with respect to chassis 4430, e.g., as shroud 4420 is depressed toward its second position to activate auto-injector 4100, or as shroud 4420 is urged back toward its first position in a post-activation, pre-lockout state of auto-injector 4100. The one or more projections 4424 may project inward with respect to housing 4402 and extend vertically along an interior surface of shroud 4420. The one or more guide rails 4441 may be configured such that a negative interior space of the one or more guide rails 4441 is similar to the shape of the one or more projections 4424, e.g., a T-shape, such that the one or more guide rails 4441 form a slot or track configured to receive the one or more projections 4424.

Referring now to FIGS. 15A and 15B, in some instances, if housing 4402 is squeezed or otherwise depressed inward, one or more functions of auto-injector 4100 may be compromised. For example, if housing 4402 is depressed inward, shroud 4420 may not be allowed to be depressed upward and/or inward to activate auto-injector 4100, or shroud 4420 may not be allowed to be urged downward and/or outward by mandrel assembly 4480 to lockout shroud 4420 and prevent needle 4416 from being re-exposed, as described above. As depicted in FIGS. 15A and 15B, chassis 4430 may include one or more ribs 4448 configured to prevent housing 4402 from being squeezed or otherwise depressed inward. For example, one or more ribs 4448 may be disposed on an outer surface of chassis 4430, as depicted in FIG. 15A, and shroud 4420 may include one or more openings 4449 configured to receive the one or more ribs 4448 and to allow the one or more ribs 4448 to contact an inner surface of housing 4402, as depicted in FIG. 15B. Thus, if housing 4402 is squeezed, housing 4402 may be depressed inward no further than the one or more ribs 4448 and may be prevented from contacting shroud 4420, thereby preventing the squeezing of housing 4402 from compromising one or more functions of auto-injector 4100. As depicted in FIG. 15A, ribs 4448 and openings 4449 may have a vertical shape or configuration, such that an upward or downward (e.g., inward or outward) translation of shroud 4420 may not be prevented by ribs 4448.

As depicted in FIGS. 15A and 15B, shroud 4420 may include one or more ribs 4429 configured to guide the movement of shroud 4420. For example, one or more ribs 4429 may protrude inward from shroud 4420 toward chassis 4430, and chassis 4430 may include one or more slots 4403 configured to receive the one or more ribs 4429 and guide the one or more ribs 4429 as shroud 4420 moves up or down (e.g., in or out) with respect to chassis 4430, e.g., as shroud 4420 is depressed toward its second position to activate auto-injector 4100, or as shroud 4420 is urged back toward its first position in a post-activation, pre-lockout state of auto-injector 4100, as described above. As depicted in FIG. 15A, ribs 4429 and corresponding slots 4403 may have a vertical shape or configuration. As depicted in FIG. 15B, ribs 4429 and corresponding slots 4403 may be disposed at or near corners of shroud 4420 and chassis 4430, respectively (e.g., corners defined by rectangles inscribed within the ovular shapes of shroud 4420 and chassis 4430), and/or alongside surfaces of shroud 4420 and chassis 4430, respectively.

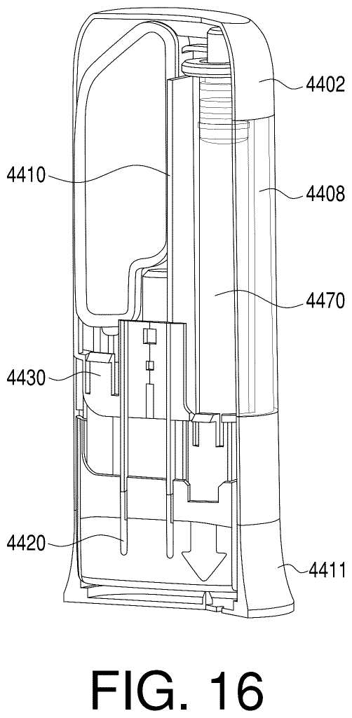

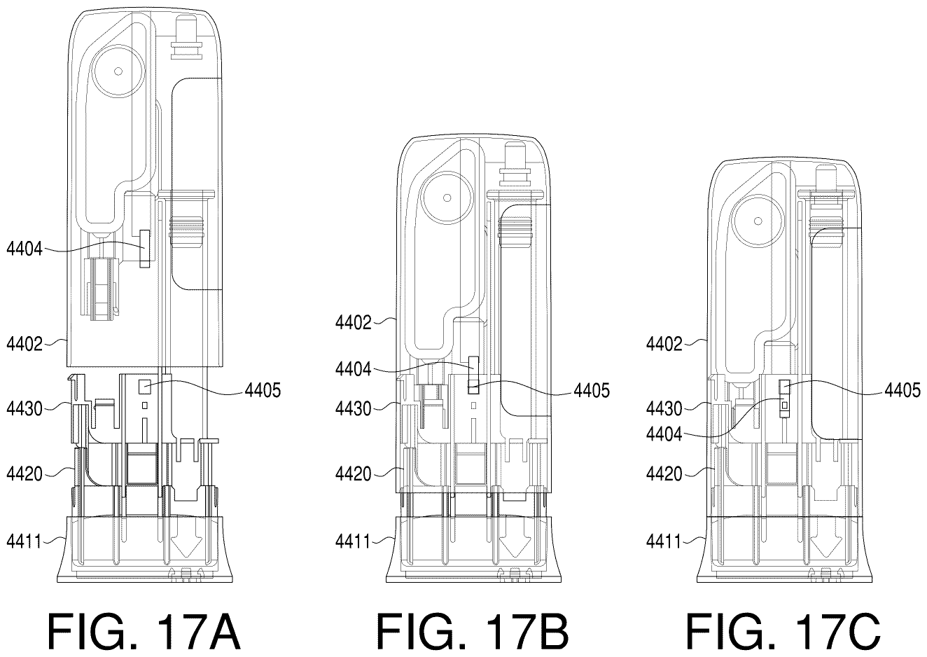

Referring now to FIG. 16, housing 4402 and chassis 4430 may include one or more features configured to allow housing 4402 and chassis 4430 to engage or interact with one another. For example, as depicted in FIG. 16, housing 4402 may include a transparent or semi-transparent window 4408 configured to allow a user of auto-injector 4100 to see through and within housing 4402, such that the user may see container 4470 and an amount or level of medicament 4472 contained within container 4470, and chassis 4430 may include an opaque window shield 4410 configured to prevent a user of auto-injector 4100 from seeing components of auto-injector 4100 beyond container 4470, e.g., valve assembly 4460 and mandrel assembly 4480. Window shield 4410 may extend vertically from chassis 4430 toward the top of housing 4402. Or for example, as depicted in FIGS. 17A-17C, housing 4402 may include a housing slot 4404 and chassis 4430 may include a corresponding chassis slot 4405. Housing slot 4404 and chassis slot 4405 may be configured to facilitate the assembly of auto-injector 4100. For example, as depicted in FIG. 17A, during the assembly of auto-injector 4100, housing 4402 and chassis 4430 may be brought toward one another until housing slot 4404 at least partially overlaps with chassis slot 4405. As depicted in FIG. 17B, after housing slot 4404 at least partially overlaps with chassis slot 4405, a tool may be inserted through both housing slot 4404 and chassis slot 4405 and used to further translate chassis 4430 upward, such that housing 4402 is brought fully down over the components of auto-injector 4100. In some instances, as depicted in FIG. 17C, when housing 4402 is brought fully down over the components of auto-injector 4100, one or more retention elements included in housing 4402 (not shown) are engaged with one or more retention elements included in chassis 4430 (not shown), such that housing 4402 and chassis 4430 are secured to one another. In some instances, securing housing 4402 and chassis 4430 to one another completes the assembly of auto-injector 4100.

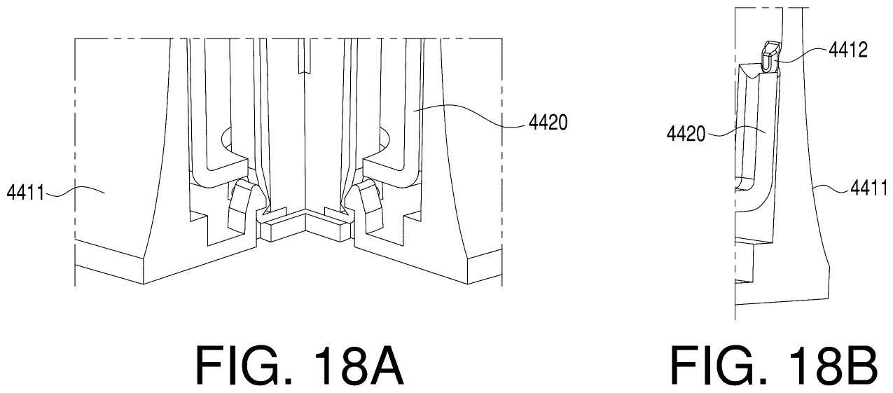

In some instances, as depicted in FIGS. 18A and 18B, cap 4411 includes one or more retention elements 4412 configured to engage with shroud 4420 to assist in securing or partially securing cap 4411 onto shroud 4420. Retention elements 4412 may include protrusions, bumps, ridges, or any other suitable feature.

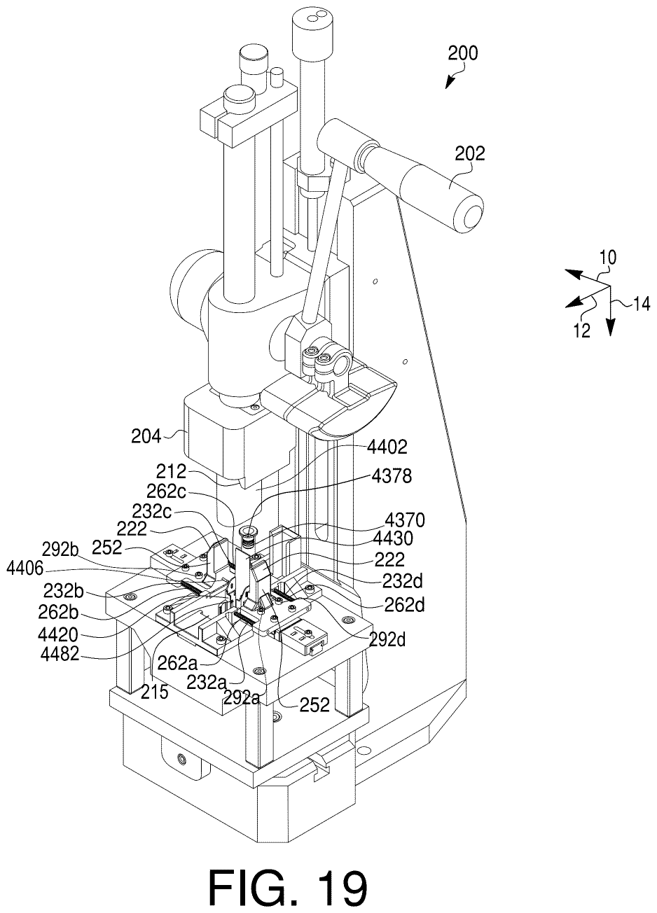

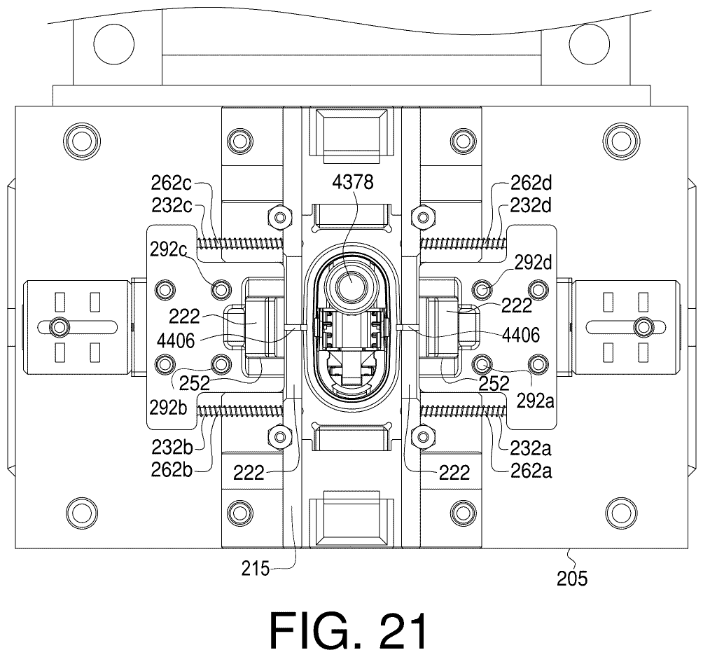

Referring now to FIGS. 19-21, an exemplary assembly system 200 for assembling auto-injector 4100 is depicted in accordance with an example of the present disclosure. Auto-injector 4100, for example, may have a length from about 0.5 inches to about 5.0 inches, a width of about 0.5 inches to about 3.0 inches, and a height from 0.5 inches to about 2.0 inches. Auto-injector 4100 also may include a grip or frictional coating such that the outer surface of auto-injector 4100 is a non-slip surface. Auto-injector 4100 may be oriented about a longitudinal axis 10 (e.g., an X axis), a lateral axis 12 (e.g., a Y axis) that is substantially perpendicular to longitudinal axis 10, and a transverse axis 14 (e.g., a Z axis) that is substantially perpendicular to both longitudinal axis 10 and lateral axis 12. Transverse auto-injectors of the present disclosure, in some embodiments, may have a longer dimension along longitudinal axis 10 than along lateral axis 12 and/or transverse axis 14. It should be appreciated that auto-injector 4100 may be sized, shaped, and/or otherwise configured as a transverse and/or non-transverse device without departing from a scope of this disclosure. System 200 may include a handle 202 configured to control the movement of holder 204. Holder 204 is configured to hold housing 4402 as it is raised or lowered based on the movement of handle 202. Housing 4402 may be held and controlled by holder 204 via a frictional coupling and/or a negative pressure coupling, such as via a holding mechanism. For example, a holding mechanism of holder 204 may be configured to generate a vacuum with an exterior of housing 4402. In this instance, the holding mechanism of holder 204 may be configured to engage and maintain continuous contact with housing 4402 via a suction (e.g., negative pressure) that securely couples housing 4402 to holder 204. In some embodiments, the holding mechanism of holder 402 may include one or more vacuum suction cups that are flexibly deformable and configured to removably attach housing 4402 to holder 204 in response to the vacuum suction cups contacting an exterior of housing 4402. When engaged by the holding mechanism, holder 204 may be configured to suspend housing 4402 relative to other components of system 200. In one embodiment, holder ledge 212 may be configured to move based on the movement of holder 204 in response to a user's movement of handle 202. In other embodiments, holder ledge 212 may be configured to move in response to an automated movement of handle 202. In the embodiment, system 200 may include a motorized lever or arm that is coupled to and configured to automate movement of handle 202 without user (manual) manipulation of handle 202. In other embodiments, handle 202 may be configured and operable as a motorized handle to move automatically, thereby automating control and movement of holder 204. Holder 204 may further include at least one holder ledge 212. System 200 may further include a platform 205. Platform 205 may house at least one brace 252 configured to move inwardly and outwardly along a horizontal axis (e.g., longitudinal axis 10). At least one depth top screw 292a, 292b, 292c, 292d (shown in FIG. 21) may be secured to brace 252. At least one depth top screw 292a, 292b, 292c, 292d may prevent longitudinal movement of brace 252 along a vertical axis (e.g., transverse axis 14). A top end of brace 252 may have at least one brace ledge 222 which may move in response to a downward force being applied on brace ledge 222. Holder ledge 212 may be configured to apply a downward force on at least one brace ledge 222. Brace ledge 222 may be configured to move inwardly or outwardly based on the level of force applied by holder ledge 212 as holder ledge 212 longitudinally moves in response to the movement of handle 202.

Platform 205 may further house at least one resilient member 232a, 232b, 232c, 232d (e.g., spring) (shown in FIG. 21). Resilient member 232a, 232b, 232c, 232d may be disposed on at least one rod 262a, 262b, 262c, 262d configured to guide the lateral movement of brace 252 (shown in FIG. 21). Resilient member 232a, 232b, 232c, 232d may be configured to resist an inward movement of brace 252 when brace ledge 222 receives a force in a longitudinal direction from holder ledge 212. Platform 205 may include a gap 215 configured to receive shroud 4420 and/or chassis 4430 during assembly of auto-injector 4100. Shroud 4420 may include one or more retention elements 4492 configured to secure shroud 4420 to housing 4402. In some embodiments, as described in detail herein, shroud 4420 may include a magnet and may be secured to a support member that is engaged with platform 205 (e.g., a rod coupling mechanism 282) using an attractive (magnetic) force, e.g., between the magnet on the shroud 4420 and a magnet of the support member (e.g., rod coupling mechanism 282). System 200 may be configured to facilitate the longitudinal movement of housing 4402 to secure housing 4402 to shroud 4420, the combined components forming auto-injector 4100. Once assembled, auto-injector 4100 may house container 4370. Container 4370 may be similar to container 112, described above.

Still referring to FIGS. 19-21, container 4370 may include a piston 4378 movably disposed within a cavity of container 4370. Piston 4378 may be movable by a pressurized fluid expelled from a fluid source. As described further herein, pressurized fluid (e.g., gas) may translate piston 4378 within container 4370, and translate container 4370 horizontally along longitudinal axis 10. The movement of piston 4378 towards second end 4130 may cause piston 4378 to act against the contents within container 4370 (e.g., drugs, medications, medicaments, etc.), which ultimately transfers force against container 4370, thereby causing container 4370 to move along longitudinal axis 10. In some embodiments, transverse auto-injectors may be oriented such that fluid source 4350 and piston 4378 are offset, or are otherwise not longitudinally aligned with one another.

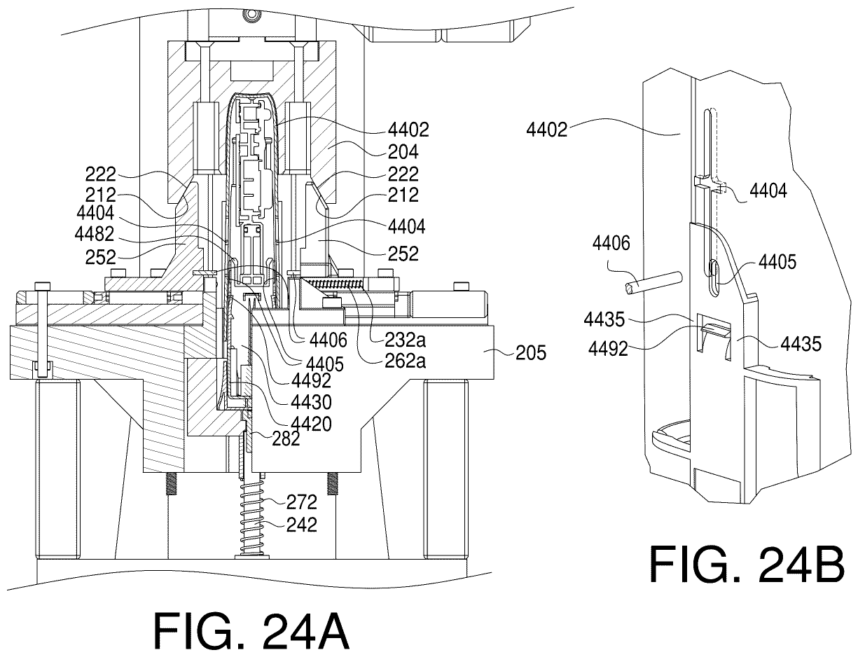

Referring still to FIGS. 19-21, brace 252 may include a pin 4406 extending from an interior surface. Each brace 252 may have a corresponding pin 4406. Pin 4406 may be configured to fit through housing slot 4404 and/or chassis slot 4405 (see FIG. 24A). Pin 4406 may be an appropriate length to laterally extend through housing slot 4404 and/or chassis slot 4405. Pin 4406 may be of any appropriate cross-sectional shape (e.g. cylindrical, spherical, rectangular) to couple to housing slot 4404 and/or chassis slot 4405.

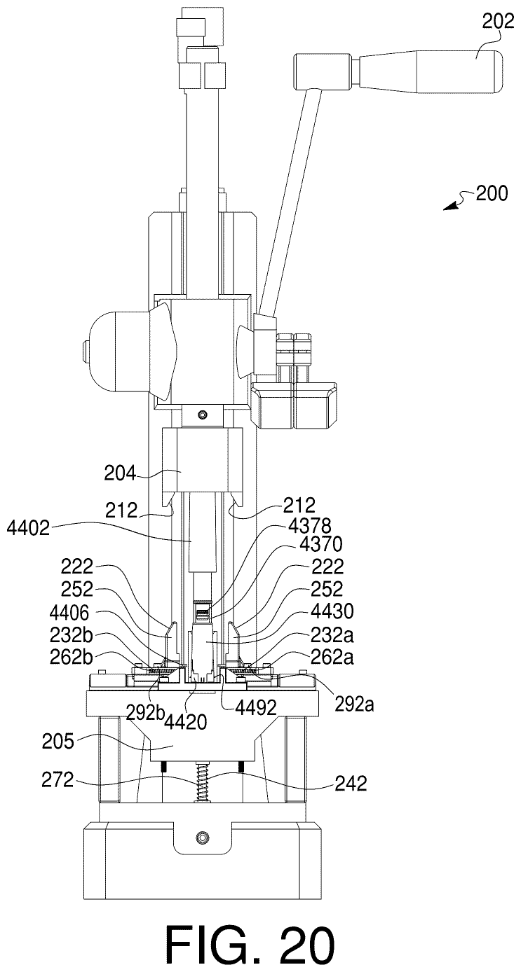

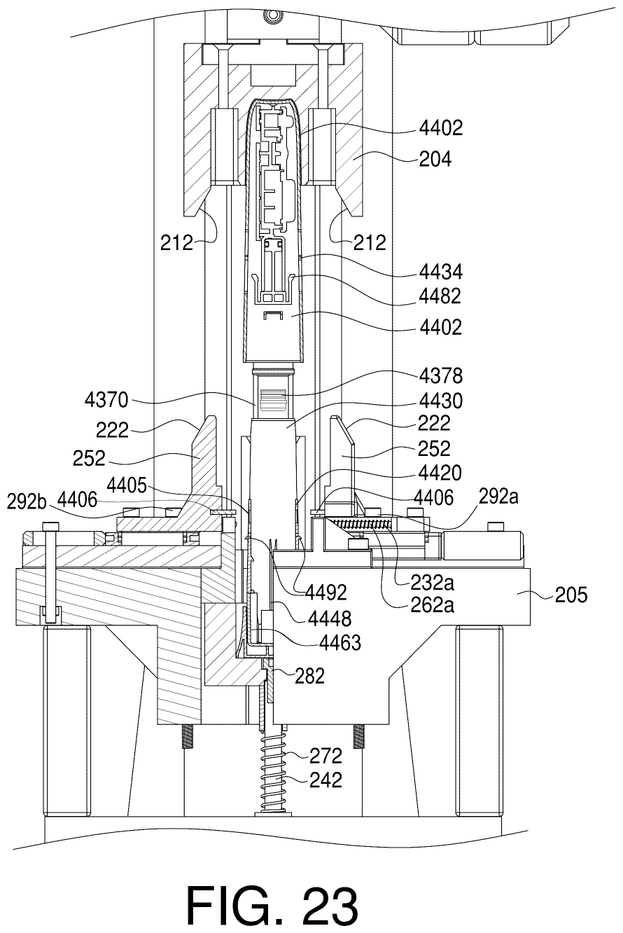

Referring to FIG. 20, during assembly, shroud 4420 may be supported by rod 272. Rod 272 may control a longitudinal movement of shroud 4420. Rod 272 may be surrounded by resilient member 242. Resilient member 242 may be a spring. Resilient member 242 may resist a longitudinal movement of shroud 4420. Rod 272 may further include a rod coupling mechanism 282 configured to couple rod 272 to shroud 4420, shown in FIG. 23. Rod coupling mechanism 282 may be a magnet configured to attract a magnetic component of auto-injector 4100. Rod coupling mechanism 282 may be attracted by magnetic region 4463 of auto-injector 4100, as shown in FIG. 23.

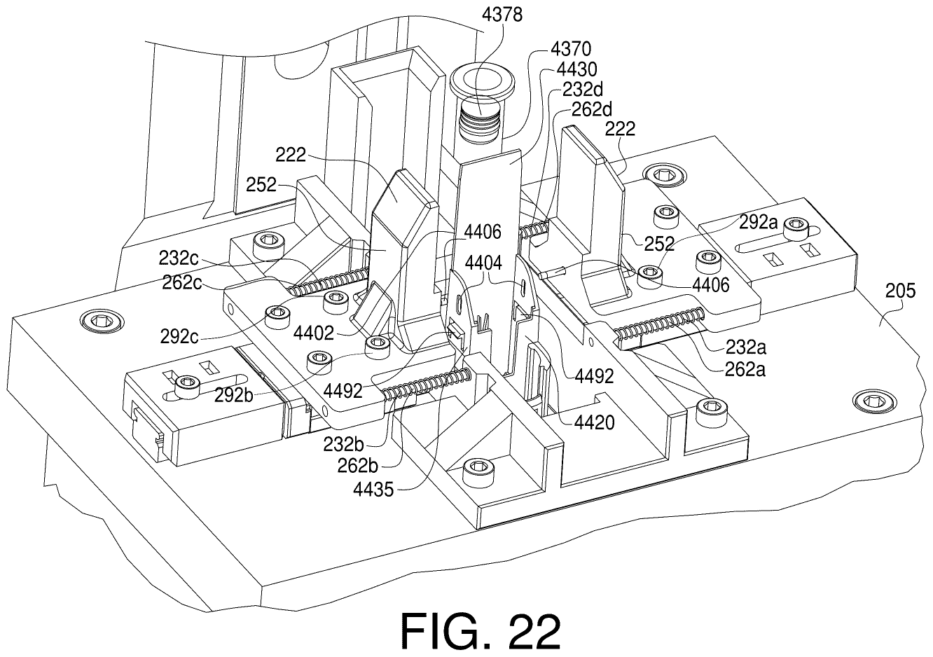

Referring now to FIG. 22, platform 205 may have a gap extending longitudinally through a center of platform 205, the gap configured to receive a longitudinal movement of shroud 4420. Shroud 4420 may have at least one guide rails 4435 disposed on either side of retention mechanisms 4482.

Referring now to FIG. 23, housing 4402 may extend from holder 204 between a first and a second holder ledge 212. Housing 4402 may include one or more retention elements 4482 configured to couple to shroud 4420. Shroud 4420 may further include one or more ribs 4448 configured to configured to prevent housing 4402 from being squeezed or otherwise depressed inward. For example, one or more ribs 4448 may be disposed on an outer surface of chassis 4430. One or more ribs 4448 may contact an inner surface of housing 4402. One or more ribs 4448 may have a vertical shape or configuration, such that an upward or downward (e.g., inward or outward) translation of shroud 4420 may not be prevented by ribs 4448.

Referring now to FIGS. 24A and 24B, holder 204 may move longitudinally downward toward platform 205 in response to a user applying a force to handle 202 and/or in response to an automated (e.g., motorized) movement of handle 202 or holder 204. As shown in FIG. 24B, prior to assembly of auto-injector 4100, pin 4406 may be spaced from one or more housing slot 4404 and/or chassis slot 4405 such that it does not extend through one or more housing slot 4404 and/or chassis slot 4405. As holder 204 moves longitudinally toward platform 205, housing 4402 may extend shroud 4420. An inward surface of housing 4402 may compress retention mechanisms 4492 inward between guide rails 4435. Housing slot 4404 may laterally align with chassis slot 4405. Ramped surfaces of holder ledge 212 and brace ledge 222 may contact and push against one another such that the ramped surface of holder ledge 212 may slide along the ramped surface of brace ledge 222. As holder ledge 212 slides along brace ledge 222, at least one resilient member 232a, 232b, 232c, 232d may compress to allow brace 252 to move laterally along at least one rod 262a, 262b, 262c, 262d on a top surface of platform 205 in an inward direction. As brace 252 moves inward, pin 4406 (on brace 252) may move closer to housing slot 4404 and/or chassis slot 4405.

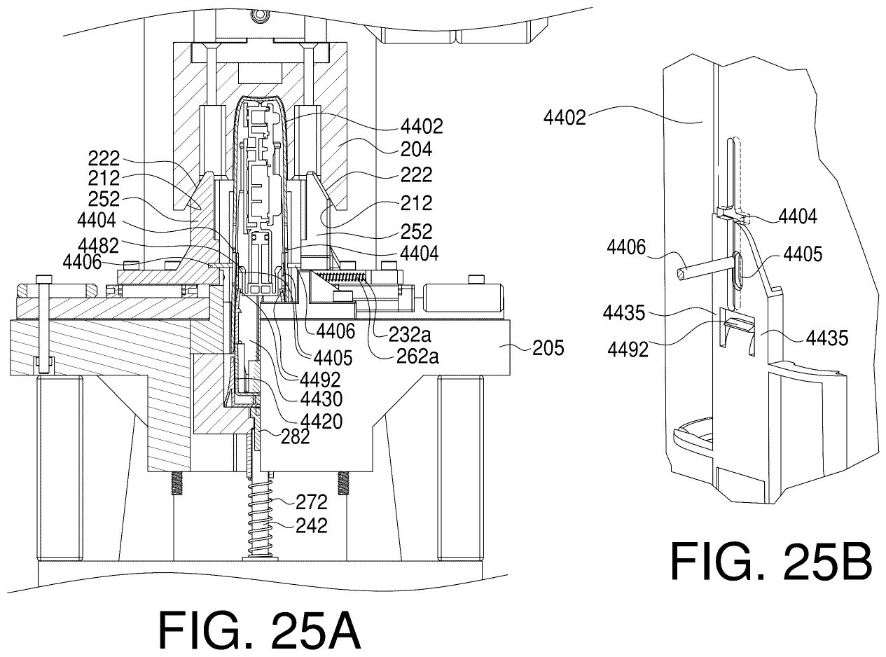

Referring now to FIGS. 25A and 25B, holder 204 may move further longitudinally toward platform 205. As housing 4402 moves closer to shroud 4420, pin 4406 may move closer to housing slot 4404 and/or chassis slot 4405. As housing 4402 may move closer to shroud 4420. The continued downward longitudinal movement of housing 4402 may cause holder ledge 212 to slide laterally along brace ledge 222. Holder ledge 212 may move longitudinally along a first axis as brace ledge 222 may move laterally along a second axis. Holder ledge 212 may apply a downward force on brace ledge 222 while holder ledge 212 slides along brace ledge 222. In response to the force applied by holder ledge 212, brace ledge 222, and therefore brace 252, may laterally translate along the second axis. The lateral translation of brace 252 may move brace 252 inward. As brace 252 moves inward as at least one resilient member 232a, 232b, 232c, 232d may further compress to allow brace 252 to move further laterally along at least one rod 262a, 262b, 262c, 262d.

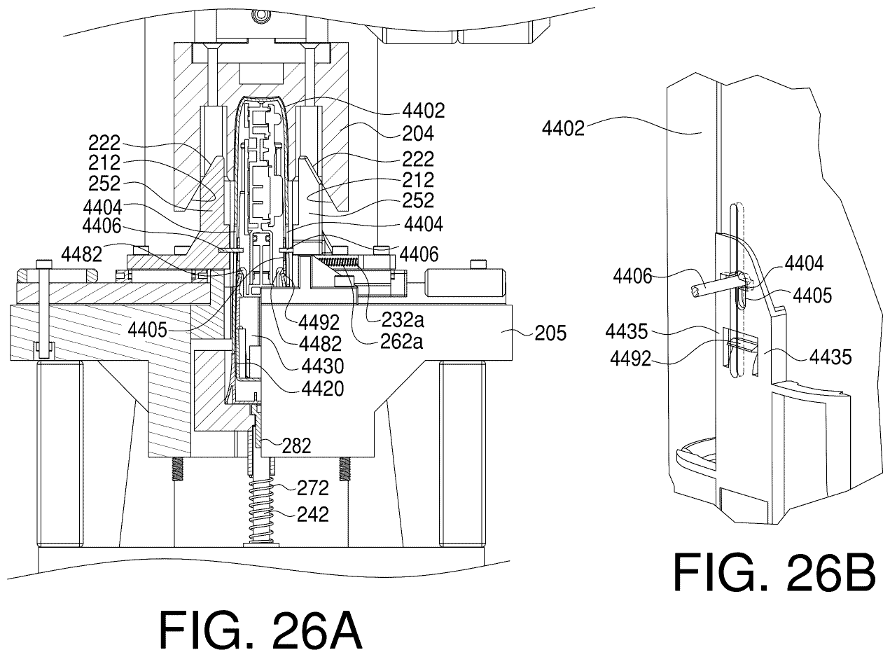

Referring now to FIGS. 26A and 26B, holder 204 may move further longitudinally toward platform 205, and housing 4402 may move closer to shroud 4420. Pin 4406 may enter chassis slot 4405 and/or housing slot 4404 such that housing 4402 and/or shroud 4420 may be configured to move longitudinally with respect to each other. As housing 4402 and shroud 4420 may move longitudinally, pin 4406 may slide along an internal surface of housing slot 4404 and/or chassis slot 4405. Resilient member 242 may be compressed as chassis 4430 moves longitudinally in response to housing 4402 applying a longitudinal force. Brace ledge 222 may further slide along holder ledge 212 to compress brace 252 inward. The inward movement of brace 252 may compress at least one resilient member 232a, 232b, 232c, 232d to allow brace 252 to move further laterally along at least one rod 262a, 262b, 262c, 262d.

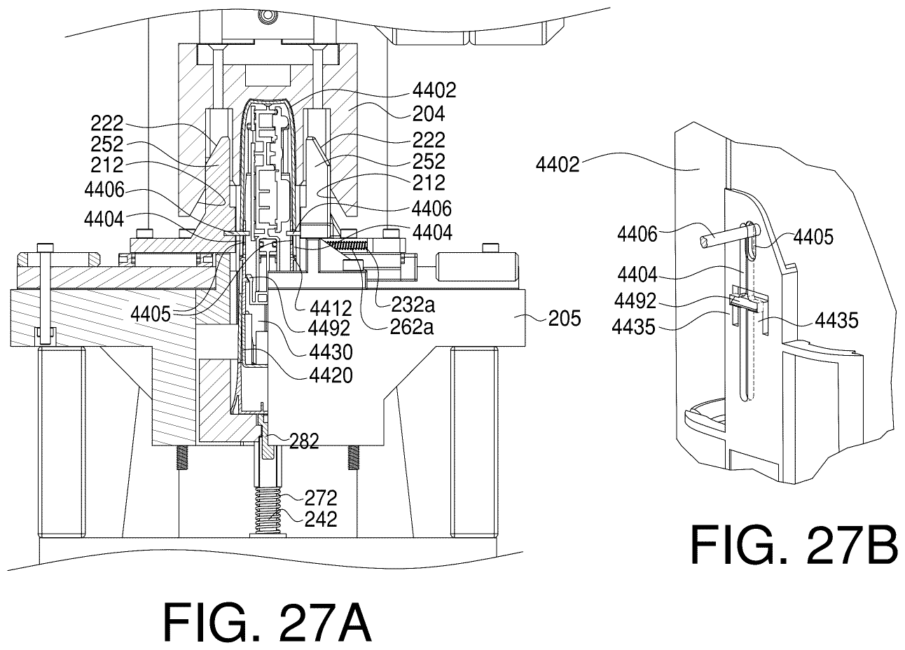

Referring now to FIGS. 27A and 27B, holder 204 may move downward until pin 4406 extends through housing slot 4404 and/or chassis slot 4405. Pin 4406 may contact a top surface of chassis slot 4405. As pin 4406 extends through housing slot 4404 and chassis slot 4405, pin 4406 may contact a top surface of housing slot 4404 such that a further longitudinal movement of housing 4402 is restricted by a presence of pin 4406. Pin 4406 may be restricted from moving longitudinally towards holder 204 by an upper surface of chassis slot 4405. Accordingly, pin 4406 may stop a longitudinal movement of housing 4402 towards platform 205, and the longitudinal force exerted by housing 4402 may overcome the attractive (magnetic) force securing shroud 4420 and/or chassis 4430 in place, thereby allowing the housing 4402 to securely couple or otherwise engage with shroud 4420 and chassis 4430. Alternatively, the longitudinal force exerted by housing 4402 may correspond to a pneumatic release of shroud 4420 and/or chassis 4430 relative to the attractive (magnetic) force securing shroud 4420 to the support member of platform 205. In this instance, a pressurized gas or air may be configured to apply the longitudinal force against housing 4402. In other embodiments, the longitudinal force exerted on housing 4402 may correspond to an electronically motorized release of shroud 4420 and/or chassis 4430, such as via activation of a motor connected thereto. Retention mechanisms 4492 may further extend through a lateral opening in housing 4402 forming a portion of housing slot 4404. As retention mechanisms 4492 may extend through housing slot 4404, housing 4402 and shroud 4420 may be coupled such that each of housing 4402 and shroud 4420 may not move independent of the other. Before retention mechanisms 4492 extend through housing slot 4404, housing 4402 and shroud 4420 are not coupled such that housing 4402 may move independently from shroud 4420. After retention mechanisms 4492 fully extend through housing slot 4404, expansion of resilient member 242 and movement of a ramped surface of brace ledge 222 relative to the ramped surface of holder ledge 212 may cause pin 4406 to retract from housing slot 4404 and chassis slot 4405 without disconnecting housing 4402 and shroud 4420. Additionally or alternatively, pin 4406 may be servo actuated (e.g., in communication or otherwise connected to a servomotor configured to provide precise control and movement of pin 4406) and electronically timed to extend and/or retract (e.g., through housing slot 4404 and/or chassis slot 4405) based on a position of housing 4402 relative to the shroud 4420 and/or chassis 4430. In other embodiments, pin 4406 may be pneumatically operated based on a position of housing 4402 relative to shroud 4420 and/or chassis 4430, such that a movement of the pin 4406 may be controlled via a release of pressurized air and/or gas. In these instances, after retention mechanisms 4492 fully extend through housing slot 4404, pin 4406 may be configured to automatically retract from housing slot 4404 and chassis slot 4405 without disconnecting housing 4402 and shroud 4420, such as in response to receiving an electronic indication that shroud 4420 and/or chassis 4430 are engaged with housing 4402. Resilient member 242 may be compressed to a maximum state when housing 4402 and shroud 4420 may be coupled. Brace ledge 222 may slide past holder ledge 212 to compress brace 252 inward to a maximum extent such that brace 252 may not move inward toward housing 4402 further.

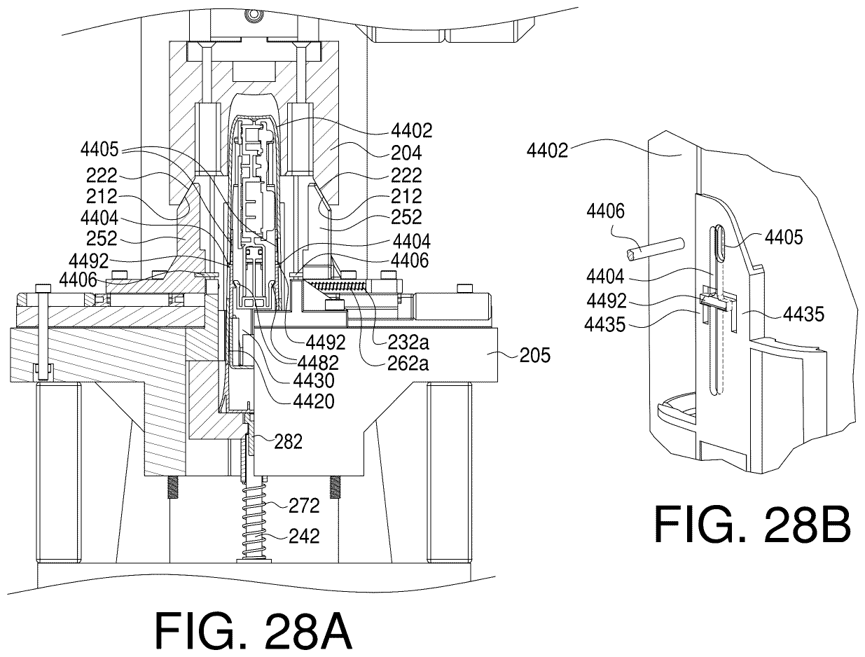

Referring now to FIGS. 28A and 28B, after housing 4402 and shroud 4420 are coupled to form auto-injector 4100, brace ledge 222 may slide along holder ledge 212 in a reverse longitudinal direction to permit brace 252 to move laterally outward. The outward movement of brace 252 may expand at least one resilient member 232a, 232b, 232c, 232d to allow brace 252 to move laterally outward along at least one rod 262a, 262b, 262c, 262d. Resilient member 242 may expand to permit auto-injector 4100 to move longitudinally toward holder 204. As resilient member 242 may expand, rod coupling mechanism 282 may apply an attractive force to shroud 4420 such that auto-injector 4100 moves longitudinally in a controlled manner. Rod coupling mechanism 282 may be selected such that the attractive force applied to auto-injector 4100 does not overpower the expansive force of resilient member 242 exerted on auto-injector 4100 after assembly. Rod coupling mechanism 282 may apply an attractive force to magnetic region 4463 of auto-injector 4100. Magnetic region 4463 may be disposed in shroud 4420, housing 4402, and/or rod coupling mechanism 282. Magnetic region 4463 may be a magnet. Magnetic region 4463 may be permanently disposed in shroud 4420 and/or housing 4402; alternatively, magnetic region 4463 may be temporarily disposed in shroud 4420 and/or housing 4402 during assembly of auto-injector 4100. Pin 4406 may move outward from housing slot 4404 and/or chassis slot 4405 such that housing 4402 and shroud 4420 may remain coupled, the coupled components forming auto-injector 4100. Following assembly, an automated inspection of auto-injector 4100 may be automatically performed to verify proper assembly by system 200. In some embodiments, the various components of auto-injector 4100 may be force monitored and/or visually analyzed using an imaging system to verify proper alignment of the components, e.g., housing 4402 relative to shroud 4420 and chassis 4430. For example, one or more sensors configured to detect an application of force (e.g., a force transducer) may monitor and measure an amount of force applied to housing 4402, shroud 4420, and/or chassis 4430 during assembly of auto-injector 4100. As a further example, the imaging system may include one or more cameras configured to visually detect and determine an alignment of the components during assembly of auto-injector 4100.

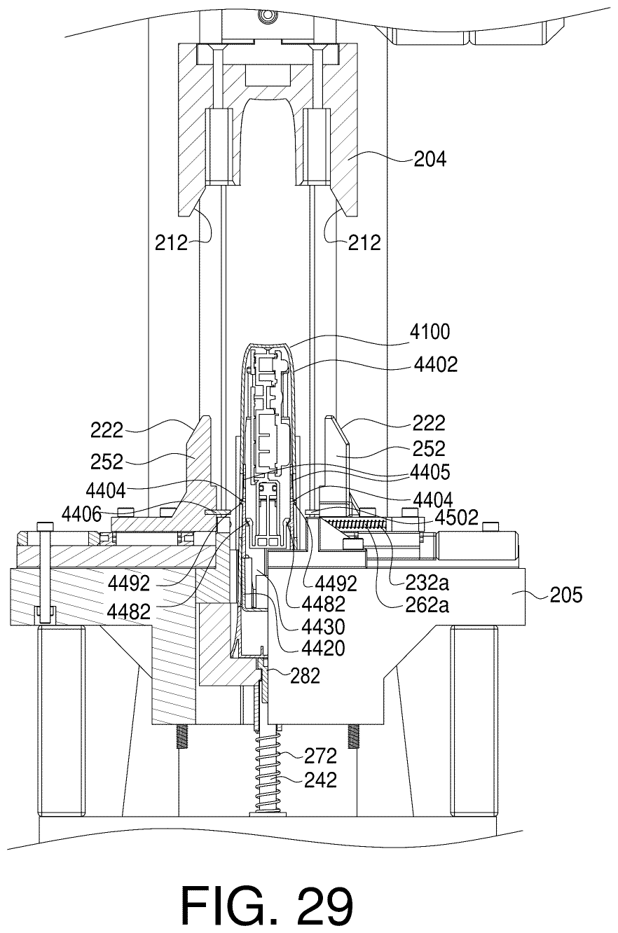

As shown in FIG. 29, holder 204 may longitudinally move to its original position (shown in FIGS. 19-20). Auto-injector 4100 may remain in an assembled position within the gap of platform 205. A user may remove auto-injector 4100 from system 200 for use.

Features enumerated above have been described within the context of particular embodiments. However, as one of ordinary skill in the art would understand, features and aspects of each embodiment may be combined, added to other embodiments, subtracted from an embodiment, etc. in any manner suitable to assist with controlled preparation and/or delivery of a drug. While a number of embodiments are presented herein, multiple variations on such embodiments, and combinations of elements from one or more embodiments, are possible and are contemplated to be within the scope of the present disclosure. Accordingly, it should be appreciated that any of the features of the particular auto-injectors shown and described herein may be included, combined, added, exchanged, and/or incorporated with any of the other auto-injectors in any suitable manner without departing from a scope of this disclosure. Moreover, those skilled in the art will appreciate that the conception upon which this disclosure is based may readily be used as a basis for designing other devices, methods, and systems for carrying out the several purposes of the present disclosure.

Embodiments of the present disclosure may include the following features:
Item 1. A system for assembling an auto-injector including a housing and a shroud, the housing including an opening, and the shroud including a coupling mechanism, the system comprising:
a holder configured to control a movement the housing along a first direction;
a brace configured to control a movement of the shroud along the first direction; and
a pin extending from the brace, wherein in a first configuration, the pin is spaced apart from the housing and the shroud, and in a second configuration, the pin extends through both of the housing and the shroud, the pin configured to maintain the housing and the shroud in contact with one another until the coupling mechanism extends through the opening to couple the housing and the shroud.
Item 2. The system of item 1, wherein, when the coupling mechanism extends through the opening, the housing and the shroud remain connected after removal of the pin from the housing and the shroud.
Item 3. The system of item 1, further including:
a first ledge disposed on the housing; and
a second ledge disposed on the brace, the first ledge configured to contact the second ledge in response to the holder moving in the first direction to move the second ledge in a second direction to push the pin through the opening.
Item 4. The system of item 3, wherein the first direction is perpendicular to the second direction.
Item 5. The system of item 3, wherein the first ledge is configured to apply a force to the second ledge such that the brace is configured to translate in the second direction to accommodate the force of the first ledge.
Item 6. The system of item 5, wherein the first ledge includes a first ramped surface, and the second ledge includes a second ramped surface; and
wherein after an initial contact between the first ramped surface and the second ramped surface, further movement of the first ledge in the first direction urges the second ledge in the second direction.
Item 7. The system of item 3, wherein the movement of the second ledge is configured to push the pin through the opening of the housing and of the shroud.
Item 8. The system of item 7, further including:
a resilient member configured to compress in response to the movement of the shroud along the first direction; and
a support member disposed between the resilient member and a distal end of the shroud, the support member configured to control an expansion of the resilient member.
Item 9. The system of item 8, wherein compression of the resilient member allows the second ledge to move in the second direction inwardly from an initial position to a middle position.
Item 10. The system of item 9, wherein the expansion of the resilient member facilitates the movement of the second ledge to return toward the initial position.
Item 11. The system of item 8, wherein the support member is a magnet.
Item 12. The system of item 11, wherein the shroud further includes a magnetic region configured to be attracted by the magnet.
Item 13. The system of item 11, wherein the support member is configured to apply an attractive force to the shroud to resist the movement of the shroud in a longitudinal direction away from the support member.
Item 14. The system of item 13, wherein the attractive force is configured to slow the movement of the shroud in the longitudinal direction.
Item 15. The system of item 1, further including a slot disposed in the shroud, wherein the pin is configured to longitudinally translate through the opening and the slot until the coupling mechanism extends through the opening to couple the housing and the shroud.
Item 16. The system of item 15, wherein the opening and the slot are configured to align to permit longitudinal movement of the pin.
Item 17. A system for assembling a medical device including a housing and a shroud, the housing a first slot including a first section perpendicular to a second section, and the shroud including a second slot configured to align with the first section of the first slot and a coupling mechanism, the system comprising:
a holder configured to control a movement the housing in a first direction; and
a pin disposed externally from the housing and the shroud, the pin configured to extend through the first section of the first slot and the second slot to control the movement of the housing in the first direction and the shroud until the coupling mechanism extends through the second section of the first slot to couple the housing and the shroud.
Item 18. The system of item 17, wherein, when the coupling mechanism extends through the second section of the first slot and the second slot, the housing and the shroud remain connected after removal of the pin from the first section of the first slot and the second slot.
Item 19. The system of item 17, further including a brace configured to translate in a second direction in response to the movement of the housing in the first direction.
Item 20. The system of item 19, wherein the first direction is perpendicular to the second direction.
Item 21. The system of item 19, further including at least one resilient member configured to control the movement of the brace in the second direction.
Item 22. The system of item 19, further including a brace ledge disposed on the brace, the brace ledge configured to contact a holder ledge disposed on the holder, the holder ledge configured to contact the brace ledge in response to the holder moving in the first direction to move the brace ledge in a second direction to push the pin through the first slot and the second slot.
Item 23. The system of item 20, wherein the pin is disposed on a face of the brace and is configured to longitudinally translate within the first section of the first slot as the housing moves in the first direction.
Item 24. The system of item 23, wherein the coupling mechanism is configured to extend through the second section of the first slot when the second slot restricts further movement of the housing in the first direction.
Item 25. The system of item 22, the system further including:
a resilient member configured to compress in response to a movement of the shroud; and
a support member disposed between the resilient member and the shroud, the support member configured to control an expansion of the resilient member.
Item 26. The system of item 25, wherein compression of the resilient member permits the brace ledge to move inwardly in the second direction from an initial position to a middle position; and
wherein when the brace ledge is in the middle position, the coupling mechanism extends through the second section of the first slot.
Item 27. The system of item 26, wherein the resilient member is configured to expand when the housing is coupled to the shroud; and
wherein the expansion of the resilient member facilitates the movement of the brace ledge from the middle position to the initial position.
Item 28. The system of item 25, wherein the support member is magnetic.
Item 29. The system of item 28, wherein the support member is configured to apply an attractive force to the shroud to resist the movement of the shroud in a longitudinal direction away from the support member.
Item 30. A method of assembling an auto-injector, the method comprising:
supporting a housing of the auto-injector with a holder, the holder including a first ledge;
supporting a shroud of the auto-injector with a base, the base including a second ledge; and
moving the holder and the housing toward the shroud in a first direction, wherein a movement of the holder causes the first ledge to push the second ledge along a second direction to push a pin through the housing and the shroud to temporarily connect the housing to the shroud.
Item 31. The method of item 30, further including:
moving the holder and the housing further in the first direction until movement of the second ledge in the second direction from an initial position to a middle position causes a coupling mechanism of the shroud to extend through an opening of the housing to couple the shroud to the housing.
Item 32. The method of item 31, further including:
moving the holder and the housing in a third direction, the third direction opposite to the first direction, such that the second ledge moves from the middle position to the initial position to remove the pin from the housing and the shroud.
Item 33. The method of item 32, wherein the shroud and the housing remain coupled after removal of the pin from the housing and the shroud.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1.-33. (canceled)

34. A system for assembling an auto-injector including a housing and a shroud, the housing including an opening, and the shroud including a coupling mechanism, the system comprising:
a holder configured to control a movement the housing along a first direction;
a brace configured to control a movement of the shroud along the first direction; and
a pin extending from the brace, wherein in a first configuration, the pin is spaced apart from the housing and the shroud, and in a second configuration, the pin extends through both of the housing and the shroud, the pin configured to maintain the housing and the shroud in contact with one another until the coupling mechanism extends through the opening to couple the housing and the shroud.

35. The system of claim 34, wherein, when the coupling mechanism extends through the opening, the housing and the shroud remain connected after removal of the pin from the housing and the shroud.

36. The system of claim 34, further including:
a first ledge disposed on the housing; and
a second ledge disposed on the brace, the first ledge configured to contact the second ledge in response to the holder moving in the first direction to move the second ledge in a second direction to push the pin through the opening;
wherein the first direction is perpendicular to the second direction, the first ledge is configured to apply a force to the second ledge such that the brace is configured to translate in the second direction to accommodate the force of the first ledge, and movement of the second ledge is configured to push the pin through the opening of the housing and the shroud.

37. The system of claim 34, further including:
a first ledge disposed on the housing; and
a second ledge disposed on the brace, the first ledge configured to contact the second ledge in response to the holder moving in the first direction to move the second ledge in a second direction to push the pin through the opening;
wherein the first direction is perpendicular to the second direction, the first ledge is configured to apply a force to the second ledge such that the brace is configured to translate in the second direction to accommodate the force of the first ledge, and movement of the second ledge is configured to push the pin through the opening of the housing and the shroud;
wherein the first ledge includes a first ramped surface and the second ledge includes a second ramped surface; and
wherein after an initial contact between the first ramped surface and the second ramped surface, further movement of the first ledge in the first direction urges the second ledge in the second direction.

38. The system of claim 34, further including:
a first ledge disposed on the housing;
a second ledge disposed on the brace, the first ledge configured to contact the second ledge in response to the holder moving in the first direction to move the second ledge in a second direction to push the pin through the opening;
a resilient member configured to compress in response to the movement of the shroud along the first direction; and
a support member disposed between the resilient member and a distal end of the shroud, the support member configured to control an expansion of the resilient member, wherein compression of the resilient member allows the second ledge to move in the second direction inwardly from an initial position to a middle position, and expansion of the resilient member facilitates movement of the second ledge to return toward the initial position;
wherein the first direction is perpendicular to the second direction, the first ledge is configured to apply a force to the second ledge such that the brace is configured to translate in the second direction to accommodate the force of the first ledge, and movement of the second ledge is configured to push the pin through the opening of the housing and the shroud; and
wherein the first ledge includes a first ramped surface and the second ledge includes a second ramped surface, and after an initial contact between the first ramped surface and the second ramped surface further movement of the first ledge in the first direction urges the second ledge in the second direction.

39. The system of claim 38, wherein the support member is a magnet and the shroud includes a magnetic region configured to be attracted to the magnet; and
wherein the support member is configured to apply an attractive force to the shroud to resist the movement of the shroud in a longitudinal direction away from the support member, and the attractive force is configured to slow the movement of the shroud in the longitudinal direction.

40. The system of claim 34, further including a slot disposed in the shroud, wherein the pin is configured to longitudinally translate through the opening and the slot until the coupling mechanism extends through the opening to couple the housing and the shroud, and the opening and the slot are configured to align to permit longitudinal movement of the pin.

41. A system for assembling a medical device including a housing and a shroud, the housing having a first slot including a first section perpendicular to a second section, and the shroud including a second slot configured to align with the first section of the first slot and a coupling mechanism, the system comprising:
a holder configured to control a movement the housing in a first direction; and
a pin disposed externally from the housing and the shroud, the pin configured to extend through the first section of the first slot and the second slot to control the movement of the housing in the first direction and the shroud until the coupling mechanism extends through the second section of the first slot to couple the housing and the shroud.

42. The system of claim 41, wherein, when the coupling mechanism extends through the second section of the first slot and the second slot, the housing and the shroud remain connected after removal of the pin from the first section of the first slot and the second slot.

43. The system of claim 41, further including a brace configured to translate in a second direction in response to the movement of the housing in the first direction, wherein the second direction is perpendicular to the first direction.

44. The system of claim 41, further including a brace configured to translate in a second direction in response to the movement of the housing in the first direction, and at least one resilient member configured to control the movement of the brace in the second direction, wherein the second direction is perpendicular to the first direction.

45. The system of claim 41, further including a brace configured to translate in a second direction in response to the movement of the housing in the first direction, and a brace ledge disposed on the brace, the brace ledge configured to contact a holder ledge disposed on the holder, the holder ledge configured to contact the brace ledge in response to the holder moving in the first direction to move the brace ledge in the second direction to push the pin through the first slot and the second slot.

46. The system of claim 41, further including a brace configured to translate in a second direction in response to the movement of the housing in the first direction, wherein the pin is disposed on a face of the brace and is configured to longitudinally translate within the first section of the first slot as the housing moves in the first direction; and
wherein the coupling mechanism is configured to extend through the second section of the first slot when the second slot restricts further movement of the housing in the first direction.

47. The system of claim 41, further including:
a brace configured to translate in a second direction in response to the movement of the housing in the first direction;
a brace ledge disposed on the brace, the brace ledge configured to contact a holder ledge disposed on the holder, the holder ledge configured to contact the brace ledge in response to the holder moving in the first direction to move the brace ledge in the second direction to push the pin through the first slot and the second slot;
a resilient member configured to compress in response to a movement of the shroud; and
a support member disposed between the resilient member and the shroud, the support member configured to control an expansion of the resilient member.

48. The system of claim 47, wherein compression of the resilient member permits the brace ledge to move inwardly in the second direction from an initial position to a middle position, and when the brace ledge is in the middle position the coupling mechanism extends through the second section of the first slot;
wherein the resilient member is configured to expand when the housing is coupled to the shroud; and
wherein the expansion of the resilient member facilitates the movement of the brace ledge from the middle position to the initial position.

49. The system of claim 47, wherein the support member is magnetic, and the support member is configured to apply an attractive force to the shroud to resist the movement of the shroud in a longitudinal direction away from the support member.

50. A method of assembling an auto-injector, the method comprising:
supporting a housing of the auto-injector with a holder, the holder including a first ledge;
supporting a shroud of the auto-injector with a base, the base including a second ledge; and
moving the holder and the housing toward the shroud in a first direction, wherein a movement of the holder causes the first ledge to push the second ledge along a second direction to push a pin through the housing and the shroud to temporarily connect the housing to the shroud.

51. The method of claim 50, further including:
moving the holder and the housing further in the first direction until movement of the second ledge in the second direction from an initial position to a middle position causes a coupling mechanism of the shroud to extend through an opening of the housing to couple the shroud to the housing.

52. The method of claim 50, further including:
moving the holder and the housing further in the first direction until movement of the second ledge in the second direction from an initial position to a middle position causes a coupling mechanism of the shroud to extend through an opening of the housing to couple the shroud to the housing; and
moving the holder and the housing in a third direction, the third direction opposite to the first direction, such that the second ledge moves from the middle position to the initial position to remove the pin from the housing and the shroud.

53. The method of claim 52, wherein the shroud and the housing remain coupled after removal of the pin from the housing and the shroud.
